SEQUENCE LISTING (25 sequences)

SEQ ID NO: 1 - DNA, 550 bp, Homo sapiens
ctataaaaaaaaaaaaaaaaaaaaaaaaaaaaaggaaagccccgcgcggcgcgggccagggaagggccacccaggggtcccccacttcccgcttgggcgcccggacggcgaatggagcaggggcgcgcaggtaggatccggggcccgcgcgcggatcgggttgcgaaggtatcgcccgggcacgccggctgagggcgttcgggaagggctcggccgccggcggggaccacggggaccaccccgactccgagcggggcccgagcccgcgactctcggtgcgcgcggagcagcgcccggttccgtccttgccgccgaacggcagcggaggcgcgaggcccggggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagct

SEQ ID NO: 2 - DNA, 53001 bp, Homo sapiens
tcttcaaaaaaaaaaaaagaatgattgacgtgcagtggcccctgccctgggaagccaaccctcaccctccaccacagcacttgatcccattgggcagtccttggcttccaccctcaaactgagagctccttgagggcagcagggggtcaaattactctctgggttctcagcactgcccagtgtgagtccaaatatctcccacctcccctgtctccgctctttcctcactcagggctccacagccatgccggccttcagcaattcctcctgcaggctgagctggttcccacagggcctttgcactagctcttccctctccctacacaagtcttcccccataggctcttcagagtctcagctcaaatggcctctactcaaaaagcctcttccagaccacctgccccactggtctaagaggtccctactttcttcttgggccctcctgacctgaaatactttgttgagtgttttccccccagtcatctggctgcatccgggttcttatggtgtccagacgttaatggtctatggggcctgtgttcgaatcccagctccacccctgctcctgcttacctgcagagtgattctgagtttctttcttttcttttttaaaattttataaatgtcaatttttgtagataggggtctcgctaacagggttgcccaggctggtctcgaactatggggttcaagtgatcctcccgcctcagcttcccaaagtgctgggattacaggcatgagccactttgcccaacccattttgaatttcttaaggactctgtgcctcagtttcctgatttgtaaaaccggtgatagtatcactcccttatagggtagttgccaggattgaacgagttaacatttgcagtgcccgacagattgtactagttactgattgaagggctgttttactatccaaatgtggctggagtaggagttgggtaaacatttattgaagaatgtgcaaccactctcacttggaagccgggctgttaggaaggggaggaggattccagtcgcccagccctcccccaccaaacgcaactgccccggcgcaaaagaggccgcggaggccaggcaggagcaggtcctggaggcctggtcggcgtgggcgttttattccgagaccaaggggatccactgcagagttctccgctgggcgtgacctcgggctacggcgtgggaggaagcgcgcggcaagacacccagcgaggtgctggggtcgcccccaggagaggacggcggctcggactgtccggcggcggcggcggggacagcgacaggggcgcgaggtggccgggacccgggccgggcgcgccgggcggggcggcgcatgcaaatctgccgggcgccggggcggggagcaggaagccggggcgggctgggtctccgcgcccaggaaagccccgcgcggcgcgggccagggaagggccacccaggggtcccccacttcccgcttgggcgcccggacggcgaatggagcaggggcgcgcaggtaggatccggggcccgcgcgcggatcgggttgcgaaggtatcgcccgggcacgccggctgagggcgttcgggaagggctcggccgccggcggggaccacggggaccaccccgactccgagcggggcccgagcccgcgactctcggtgcgcgcggagcagcgcccggttccgtccttgccgccgaacggcagcggaggcgcgaggcccggggtacgtggacacccagcgctccccaaagccggtgctggcagtgagacctccgccgggacggcctgcgggggtggggggcttggggttaggctgtcggaggccacgcagcccctcttctccgggcagtggcgcccagccctgcgctcaggaagtcagtgaggacttcggagagagaaagggtggggaaagttctgagaactgtaaatttgagtagtaggtcagtgaatcggggcggtctccgcctccgaatatcagatggacccaataatcgcgatcattgactgggacttgttttactgaaaggatgccaagtgaaacctcccactaacttctctgtggccggtgctgtgcgtcatggacattgtacagatgaggtcaccaagaccccagccgctttagtaacttgccctaaggtgccgcccacttgggagtccgtggggcagcaggactagagcccaggcaatcccacgccagagccagctgctctccatctctcaatcacgtttgaggagcccccacaataaccagaatctccgggagatttgcttaaaatgcagattcccaggtccttgccctgagattcagattcagtaaacccaggaatctgatctttattttatttttaaaaatttattttataaagatggtgtctcactatgttgtccaggctggcctcaagtgatcctcccacctcggcctcccaaaatgctgggatgacaggtgtgagccactgagccctgattttttttttaaacaaaatctggatttcttagacttaagcaagataggggaccactagtttgtctgcttctggggttcaaggaatgcccccaggccagcgctaaccctgctccagttctagtctctccccttccccagccctctgggaggcatgacccactgcacttccctgcccccagtagcctatgaggagcccaaggcatttgctgggacctcagtgcctttatcaaaatcccaaagtccaatggggaaacaggctcaggtgtaggtttggccagggtgtcctgcacattagggcattccagggtcctgcaggggtttcttagaactcggctagcccagcccatgaatgggaggtgacctgctaagccactcattcactcactagcactcattcagctcctgcgaggccctgggtggtgttctgggcactggtgcttgcctcaaggagcaccccctctgcaggaagacagacgtgagggctgggagggtgcggatgcaccagcggctgcaggaccacaagccagaggggatcagctcttcaaaggtcactgggccaggcttcccagcacccagggaccctgagctagccttgtgagtagagcaggggagtctggcaggtagaggatgggaggtaaagggaggcacagagtgggcggaggctgggaagtgtgaaagggcactgtgtacagaacctagggagtaagcagaagccagagatggatggaaagttgggctgggctgcattgtggaaagccttgaatgccatgccaaggaacttgggccagtgtattcaccttggcttgagttgtaaaagcaacaatgcagattcctgggccccaccctgggcctctggatctgaatctggagggagacgctggggaggcagcagggaggaaaacctgcagtcactcaccccatttacaacacatgcagttaccggcaggtccgagataacctaagggtttccaaatgctaggaagtgactcaatgactgatatgtttaatacaatacaatcccggttgcaaaatgcccagggcaagggtgggggtgttcagttttctaatgctcccagttcaaaaaggagctgaaatgaattgtcaaagaagtgacaagctgggcgcagtgactcacacctgtaatcccagcactttgggaggctgagagaggatcgcttgaacccaggagtttgagaccagcctgggcaacatagtgagatcctgttcctacaaaaaaaatttaaaaacttagccaggcgtggtagcaggtgcctctagccccagctatttgggaggctgaggtgggaggatggcttgagcccaggtggtcaaggctgcagtgaactatgattgtgccactgcactgtagcctgggtgacagagcgagactctgtctcaaaataaacaaatacataaatacatgagaagagagaaccttttctacctcccccatctccctgccctcttccacccaccccatctgggacgatggaggagagcgtttgggattagatgccccacctctcgactgccctcactggtacctgggagtgaggccaggacagaagccgcctaagtactgttcttcctcacatttgtaaaagtgaactttcttgagggttgatgacatgttttataagctacttcacacaatgtccaacacccagtgggtccacaatcaatgtatatgtctgttgttacctgtcaaaacgtatagctggggagtgacagtatggacttgagccagatcacccagtgcaaatgctggctttgctgcttactgtgtaactgacttcccagagcctcagtttctgcatttatgagattatagtgactatgccattgtagtgatgactaaatgagttaatatatgtcaagcatacaaaacagtacctggtatgtagtacaaactatgtatttgttaaatgaataccttttttttttttttagagccatggtctctctctatttcccaggctggagcacagtggcacaatcatagctcactgtagcatccaatacctgggttcaagtaatccttccacctcagcctcctgagtagctgggactacaggtgtgtgccaccacggctggctaattgtttttatatttttagagacaggatcttgctatgttgcctaggctagtcttgaactgctggtctcaagcagtcctcctgcctcagcctcctaaagtgttggatttatagatgcgagcaaacgtgcctggcctaaatgaataaatctttactgttgcctcctttgagctttattggttgggatttagtccactcattttgcaggtgagaactgagacccggagatgaatgcagtagccttgcctcaggtccacatctgagggacagactccatcctacttttcttcccatgtattctttcctgcttgtaccagttcactggggtgaccaagaaagacatttctgagttttcctcctggctggcacagtggagacatgcccagccacgtttagctagacttaccatggctgggctagaagagaggccaggagcttgtctggaggttgcacagacctgcccttggtgaatgtggatcggagcgccccggcagaggcttgaggtgctacagaggctgggggatccactgtcagaccgggtgcatcaccactgttttatgatatccagcaaattgctctgcgtctctgagcttaaggcagagagaagggcctgagcttgaagaggggatcccaggtttaaatcggagctggctacttcccagctctccactgtgtaatctggaggaagttgtttaacctctctgagcctccctttgttttgtcatgggtgtttgcagggagagcattcatgagatagtttgtatagacattttgcccttggaaggcgctcagtgattgctgtttttctctgcattttggaccgagtcccttctgcgtatccagcacctggcttcctcctgcatttcttcctcaggaatttattgagcacctatatgtgccagagcatcggaccaagtcctttgctctcctggggtttagattctagttgagcatacaggcaatgtacaaccaatacatatgttcagatcggcagctttatctgttattctgaaatccacgatttctgaaaaccgaaagtttttcataactcattttgaagctaaacttgaggctttttagagtcttcatcctcttcggtgtgatgctcagatgtctttctgcagagatattagtgcaattgatctttgggtgctttcccgccctgcatgatgggcatgtcggtgggtattttatacaaggtgacctttctacagcctgagtcatcctgaattctgaactatgtctggcccccaggattttacaggagagaacatggacctgcaatatcctatcaggtggagactgtgctaggaaggaaagcaaagctgggtgcaagtggaatcagggagggcttccctgaggaggtgacatttgagagggatgaggaagagagggccaggtggagggactcacagaggacggaagactccacttcctgactggcctgtgcttacccctcattctgcatgcacagttctgtctaaatgcgtccgttcttgcctttccaagaagtgctgatgtctcttacgtttcaagtctagctctgactgactcattctctctttaggtgcccccacaggcctcctcacctcttctcgtttgcttaagaaatctgaggtctgcgctaaagccagcctgggtgctgtaacctctcggccgaggatggtgcccagctgggcttgcatctggagttggaggaagtgggtcccgggaggggaagaggagccgtggggccttccaagggtttggtggaattgagttcctggtggggaggggctgtgttctgggtcttagagcctctcgcctgcaaagcaaagttgaacgtaacacgcgctcacatatctcgagtgccagcccggggctaggtgcttaacgcttgcttagccaaagtgccaggggctggggacacgggaatgagtgacccagaaatgagggccctcatggagctgaaagcattctgggcttggtgactttgaggaagtggcttttgaactgagacctgaaataccaaggcatgtggatatctggggaaagagtgatcctggcagtgggaacagagagtgcaaaggcccagaggcatgaatgagcttggtgtgtttgacccacagcaaggaagctggtgtggttggatggaaccaaacaagaaagaggtctaggagcagaggtcagggagggagctggggccagatacctgcattgtctcatttaacttgtgtaataaccctaccagataggagctactctcatgcaaatgttacagatgaagtaacagaggcacagagaggttcagtgacttgtccagggtcacacacctgaaggtggcagagcagggctgtgaacctagaagctccagagtcctcagtcctcaccatggtgtttaaacacaataaaatctatgcctgagtgctgaagacacacctgccatttagatcgatccacttttttacattgtcggtgtactccctgccatccatctgcccagaaatactgtgaatgtgggcagcgcaggactggttcctccaagccccattttacagacagtccaatcattttacatattattttatgaatgagcaccttcctcagtgtctcagaccaggggtccccacacagcagggccacacagcaggaggtgagtggcgggtgagtgagcgaagttttatctgtatttacagctgctccccatcgctcgcattaccacccaagctcggccacctgtcaggtcagctgaggcattagatgattctcacaggagcacaaacccttttgtgaactgcacatgtgagggatctgggttgcgtactttttcttttttttttaagacggagtttcgctcttgtcgcccagactggagtgcaatggtgcgatcttggctcactgcaatctccacctccccatgttcaagtgattctcctgcctcagcctggtggctgggattacaggcctgtgctatcacacccagctaatttttgtatttttagtagagacagggtttcactgtgttggccaggctggtgtcgaactcctggcctcaggtgatccacctgcctcagtctcccaaagtgctgggattacaggcatgagccactgcacgcggccctgggtgcgtgcttcttatgagactctaatgcctgatgatctgtcactgtctcccatcgcccttggatgggaccatctaattgcaggaaaataagctcagggctcccactgattctgcattatggtgagttgtataattatttcattatatgttaccatataataataatagaaataaagtacgtaatacatgtaatgtgcttgaatcatcccgaagccatggccccaaccacggcctctggcagtggaggctggagtccacatcacgctctgtgctgggtgcaggaatggagatgagtgggtgtcctgctcatttccctgaggaggaacctgaggctcaggctagagacttacctgggtgacacggctcctaagtggcagagtccagattcaaacccagggctacagacccagaagcctgtgcatttaaccacggttctgagctatccagcccaaagaggagagtttaaaaggattacatttcttcgttggaaatcagtgagcatagtcgcccagaaccatttttgaaaaactcagagaaggcctatgttttcctgaagcggccagggaagtattctgcctttttaaaagggaagactgaattctagggaggagaggcaccagctctctgggctgggcaggtctccaagctggcagggccacagcctcccgacggtcttgcctctagcatatttcttccagaagccacatttccacaggtgggcagctctaactgggagaaaatcttcctgagatttagccaaattagctttctttctttctttttttttttttgagatggagtctcactctgctgcccaggctggagtgcagtggcgcaatctcagctcactgcaatctctgcctcccgggttcaagtgattctcctgcctcaacctcccgagtagctgggatttcaggcacacaccaccacgcctggctaatttttgtatttttattagcaatggggtttcaccatgctggccagactggtctcaaactcctgacctagggtgatccgcccacctcagcctcccaaagtgctggtattacaggtgtgagccaccgtgcccagcccaaattagctttctaggagcttccactttctgagtctgtttctgcctcttggaggtagtgaccatctccctccaaactcggatacttctcttattcagactacacaactgtagttatttcaggtattctccctgactcaggaaattataagtttggcctgccttggcacatgagtcaactggttggagtcccacgcaaagactgtttgtttggtcaaagaggaaggagactattacctccttggatctagagcttatacttctgttgatgcaacctggaatcatactgtgtagcagccatatcccacttggagtgcatattgggcttgtcagctaaaatctccatgtttctcttctatatgagctgctgttaatccaagtttcttctattctgagagtccttgtctaaagctaagaggttccagtttttcatgctctctaataaatctacttcaggctaacatgctatcattttttctttgagtactgctttggtttctttccacaacttgttacacaatgttattcactgcccagtttagaatttcccttttggttcccattttaagacttatttaattatatgctttttaaatttttgagacagatagaattttttaagttatctttttattgttgatttctaaatttggtcatcatggttgttgtggtcaatatatgtggttcctataatatcagtggtagagaatttgctgagatctttataggattttctggtcatttttttcctcttgtgtttgaaaagaatgtatattttctgttgggtataaggttctagaaatgtatctatttgatcaagctagctattatattatctatatttgttatgtcactatgtggtttttttttttttacctgatttacctcccaccgtaatacaattacaattcaccataatagaggctttgttaagttctccttttgagtctgtcagttttttgctttatttttgaagctatgttgttaagtgcatagccatatgagtagaataagggttcaggatttttatacctttttggtgaattgttgcattttcccgagtagctgggactacagatgtgcactaccacgcctagctgatgtttgtgttttttgtagagatgggatttcgctatgtttcccgggccagtctcaaactcctaggttcaagcagtctgcccacctcagcctcccaaagtgctgggattacaggcatgagccaccgcagctggctgaattgttcctttaatcagtataaaagagccaacttccagcctgggcagcatagtgagaccccatctctaccaaaaactaaaaattagctgggtgtggtggcgtgcacctgtagttccagctacttgggagggcgaggtgggagaatcacttgagaccaggaattcaaggccgcagtgagctatgattgctccactgcactccagtctaggtgacagagtgagacattctcaaaaaaacaaaaacaaaaaaaccatccaccttctctcttctaatacttactcaagacatcattgtcatgaattctattttattgatgtcagtactcctgtactaataagccagatgagttaaaaacaacagcagccttgcaaccagccatgaagcaagtgatcttattctcatttcacagatgaggagactgaggctctgagtggactccagcctgccacggtctcccaccgagtcaggttggagcctgcacttaggcccagagttatgcttctgtgtctcagatgcttgggggtccctgcccacaaaggtggcccccatgtctgtgtgtccccacccatccaggaatggtatggggtgagacaacaggaagagctccatgggtagtccccacccactgccttccatatgccagtctgcaagcagaaaatgaacgggaagctcttacctgtgcccatgtgttgtcctggctttgggtttgcccagggggcaaagtcctggtcattgttgagacaagttcactgctgctgagggccgagcaaatggtgacaaaggccagattctaaattcctacctggttacttcctgctccgtcaccatcacctcgggcaaattctgaacctccctcagcctcattcctccatctgtgcttgcagggagaatattttactgtatcttccatagtgagctttccaggagaagcaaaaaagagtggtgtataaaaaagcaagtttatgactgcttctgtgccaggcactgtgctgtgcactccagagccaaaggcctgggttcaaatcccagcccactcactagctgtgcatccttaggcaggttcctgaccctctctgggcctcggcttctccatctctgcaatggagatgatattaattgtatctgcaatgctccttggattgggtccatggttaagtgagtgagtgaggctcaagggcttctttttttttttttagagatagggtctcactctgttgcccaggctggagtgcagtggcatgatcacagttcactgcagctgcgacctcgtgggctcaagccatcctcctgcctcatctcccttagtagctgggactacaggcatgtgccaccatgcctggcaatttttttttgtatttttggtagagacggggtcttgccatgttacccaggctggtcttgaactcctggactcaagtgatctacctgcctcagcctcccaaatggctgtgattacaggtaggagccaccgcgcccagccttcaagtgcttttttttgttagtgagactggtacaaccaatgccaaggctggtgagcaaagttgcacccagttggggaggggcgaaaccaagggcagaagggcccagggcatggcctctcatggctgaaggaagaggggacacgtggccagtcacagtgacctctgcagacctcaagaaggaggccagctaggagccatagctgtcactaagacagtcaaaatctaatcagcaccctggcgccaagaggccattgtccgctgctcaggacagtgagtgatagtcgataaccagctctctggaggtcagggccccgaggtggctgagatctgtgcctgcattaggacatttaaggggactccaagccaggatttgctagtgtttgcccagcagggagaggtggaaaattggggatgaccaaccaggcctccctgctcacagccaggggaattcatcctgtcgttcttccattcagcaaatattcatggagcacctgctgtgtgccaggcaccgtgctaggctctggggagatggctgtgagcgggatcgacactggccttgtgtgagctggccttgcagtaggggatatgagcaccaaacacataaggcccgagtgtgcaatatgccacaaagattgcagagaaataaagcaagataacaggttgcagaaagatggaggccaggctggctgctgttcctgggtaggtggcttctggacacagtttcctcaggctctgtttcctcctttgggcctctggaatcttctgaagacaggcctggcttccaggatcccggatggagttgcctgttgactgtgtggactcaaagtgtccacagtgcgccggagcgcgtggtcttccccagcaaatctgcttctcttctcggctctccatcgtgggaagggtccctgggatcctccccaccaccccagcctgaggccactttccctctgtccccgctctccccagcctgctttccccaccaagcgccaccaagtccctgctgcctcctcctcccaaacaggctcaaacctgccccctcttcgccagctctcagctcagcccgctcctctcctgcttaagagccttcactggctccccactgccctagagacagagtttgagtctactgccatggcttggcagacggtgttgtccccggcctgccaaccttccagctgccatcccagccagtccttctcccccttgctccagccaagggccccgcttgtagggtgggaagtgggccgtgccctccctcctgcctgcctattcgctgggtcttccctcattccctccctcaccccgccctctcataagaaccttcagaatctgccagacgcagtggctcacgcctgtaatcccagcactttgggaggctgaggtgggtggatcacctgaggtcaggagtttgagaccagcttggccaacatggcaaaaccctgtgtctgctaaaaatacaaaaaaattagctgggcgttgttgtgggcacctgtagtcccagctactcgggagactgaggcaggagaactacttgaacctgggagacagaggttgcagtgaactgagatcccaccattgcactccagcctggactacacagtgagactctgtctcaaaaaaaaaaaaagaaccttcagaatcataaaatgggtttaataattatacttatctcatagggagggcttaaagcacctaggccagtgtctggcaccaaggaaacactgtatacctgttaaaataagaataaaataaaatacacatgctcacttattgagtgcctgctgtatgtcgggcccctccccgagagaactctggtgcatccccgataaagaaacctgaccctctaggatgagggagggtcttccctgagcgcctgctaacagcctcctctgctaatctgaagtcctgcagcttgcccagcaggctggcttgtcacacgggctacccccacaggccgccatcttggaaaaggccaatggctgtggaatcattcttgactcctattagccttcctgttctctctcccgccccacagcatccatcctgtcagcaaatcctgtgggctctgtcttcaacacagatctagaattcagccccttctcaacctccccatctccgcagcctggtgtgagccctggcacctctctctggggccagtgtggtagcctcctcactgctacccacttctacccgtgcccccaccacccaagcaagagaggtcttttttaaagtctcatcatgtcacttctttgctcaaaacgctctggcaacttcttccttgctcaaatatttgttaaaagaagaaaggaatgaatgaatgaatggcctgtttgcatctgtaatagccagcacataggcagtacttggaaaacacccacagcaagaataaatgaggagatggctggacacggcagctcatgcctgtagcatgttggaaggccgaggcaggaggtttgcttgaggccaggagtttgaggttgcagtgagctatgattgtgccatagcactccagcttgggccacagagagagatccccatctctacaaaaatttaaaaattattcgagcatggtggtgtgtgcctgtagtcacaactacttgggaggctaaggcaggaggatcacttgagcccaggagttcaaggctgcactgagccgtgacggtgcgactgcattctagccacagaatgagaccctgtctctaaagcaaaaaagagaaggagaataagtgaggtacttgagcaagtgggtgaggatggttgagccacagcaggcccacagggaggctggtgggtgctgttggtggagggggccgatttgtgttcctggtggctgcagagccagtctgttgcctcccctccctcccactggctgggctggggacggagccaaccaggcagatcccgggccattctggggccctggcctggcctgccttactatcccagtggggccaccgacgtggctgccatttctggaaaccccagttctaggtaggttactgtcccaggaagaagtgcatgcctagcttcctgccagcccagcccagctgtcccagaagccagaggaagctctgggctggtttcttataagtagcatgactaactgattgcgccacaggagctgaggtttcaggccagtttctgtcgtggagtttcaggggtcagcggctccaaagccccagcccccacccgaggcccctgctgtccctggattcctctccacatgccacctgtcacagaggtgcttgcatcctgtctgcagaaccagcccctcctccaagccaggtttcctgcccagagggaagaagagcagaaggcctgtaccgccccctgtgcgcccccagtcctccgccccctgcccccctgccacccgctgtgggttcgggaagagtcctgctgggtcgcttccaggctgctgctggcttctgacatttcaaggcttaatcacttaatctcccccgagttttttaaaaaaagtgagtcaagtgtgttctcctggtggcgggcgtgttttcccaggcccatgcttctgctcatttcctggctgcggccttcactgactttgctgcttcctaactctgtgaccttggccttgtcacttgctgtctctgggtctcaggccaatgggtctcaggccagtggggctccccggacaaagttccagggttttggggactgcatttcaagggtcagcaaccacagtccttaaacacgtggattttggagtgacaatgcctaggttatggttctgtgaccaccagcaagtgctctagcctttttgtgcctcagtcttctcatcagtaaaatgggaatgttaatggcacctacctcatagggtcgttgggagaaccaaattgattaatatattgtaagtacttagaacagggccaggcacaaaataagtactgcatatacagttaactatggacaaaagcttgggacctggttcatgtttaaaaatgattattgaggccaggtgcggtagctcatgcctgtaatcccagcactatgggaggctgaggcaggcggatcaggaggtcaagagatcgagaccatcctggccaacatggtgaaatcccgtttctactaaaaatacaaaaattagccaggtgtgatggcgtgctcctgtagtcccagctactcaggaggctgaggcaggagaatcacttgaacccacgcggaggttgcagtgagctgatatcgtgccactgcactccagactggtgacaaagctaggctccatctcaaaaaaaaaaaaaaaaaagattgttgaatttattattgctgaatttttttttttttgagacagaactttgctctattgcccgggctggagtgcaatggtgtgatctcaactcattgcagcctctgcctcttgggttcaagcgattctcctgcctcagcctctcagcctcccgagtagctgggaatacaggcacccaccatcatgcccggctaatttttgtatttttgtagagatgtggtttcaccatgttggtgaactcctgacctcaggtgatccacctgcctcggcctcccaaagtgctgggattacaggcgtgagccactgtgcccagcctattattgctgacttttaaaccattcattcatttaatatatgtttattgtacacatgctatgcctgtgtcaggcactgtttcaggcactggggataaagcagtgagcacagcagacataaagctctgccctcatggggcttgtggtctctcagagggaggcaaacaatgcatttatagacaaatacataatttaaatttgaggttacctattatactcccaacttgaaaaactagcagtatattgaggtggctatttcacatctgatggaggaatatgtcttttgtgaaccatttatgttctttgcctatttttcttaaagggatgtttgtcttattgatttgtaagaactcttcatatattaaggccattaaccgttcataatgtagattgctacctttttctagtcttttgcttgctgatatagttgggatgtcatcctctctaaatctcttgttgaaatgtgatccccagtgttggaggtgggtcctggtgggagatgttgggtcatggggacgggtccctcatggtttggttctgtccttgccatagtgagtgagttctcacaagatctggttgtttagaagtgtgcggcacctccctcctctctcttgccctggctgtcaccatgtacgcctgctcccctcttgccttccccgtgaataaaagctccctgaggcctccccagaagccaagtagatacgggtgctatgcttcctgtacagcctgcagagccataagccaattaaacctcttttctttgtaaattactcagtctcagatatttctttatagcaatgcaaaaatggactaacacacttgagttctgtctttttcttatggcatattttaatctaggccagacacagtggctcatgcctgtaatcccagcactttggaggccaaggcgggtggatcacttgaggtcaggagtttaagaccagcctggccaacatggggaaaccctgtctctactaaaaacacaaaaaattagttgggtgtggtggcacacacctgtaattccagctactcgagaggctgagacacaagaatcgcttgaacccgggaggtggaggttgcagtgagccgacatcacgccactgctctccagcttggggaacagagcaaacctccgtctcaaaaaaaaaaaaaaaaatctagggaagttgaaaacattttgcagtcaaagctatctttgctatctgcatcttgaaataaaataaacattcatacatgtaacctcattaatctgattagagatagtgataagtactgggacagacataaacagggtgctgtgataagaagtcacagggagggcccgcattagttgaggggggtcagagaaggcttctgaagtgtttctggtgcaaatgcttggaaaagggagagctgggagtgtatcccagggctgaggccaatggaggattgcagaagagggagaggggataggaggggaatgttagagggataagcaggggaattggctcttttcttcattttcttaaattaaactttttattttcagataattaaagatttacacacagctggaagaaatcatagagaagccgggcgtggtggctcatgcctataatcccagcacttttggaggctgaggcgggcagatcacttgagatcaggagttcgagaccagcctggtcagtatgatgaaaccccatctctactaaaaatacaaaaattagttgggcatggtggcttgcctgtagtcccagctactggggaggctgaggcaggagaatcacttgagcctaggaggtagaggttgcagtgagccgagatcacaccactgcactccagcctgggcaacagagtgagactccatctcaaaaaaaaaaaaaagaaaagaaaaagaaaaagaaatcacagagagacccctgcttctatgctgttccccccgagggcaagattgtagaaatctgtaatacagtatcacagctgggatactgacactgatataagctactgaccttattgggatttcccgatttacttgtactcatttttgtgtttgcgtacgcatgtgtggtttagtgctatgcaattttatcacggcataggtttgtgtatgcagccaggatccagcacagttctatcatcacaaggatggctcaaattctccttttatagccacatccaactcccttcctgacccaccccctcagcccctggcacccactgatgtgttttccatttccatggttatgtgatttcaagtatgttacataaatggagtcacggggtgtgtactctgggaactggcttttttcactctagggcttggattttattctaagtcagagatgcttctctaatgagctgtgaaccccctggggatctgtggcccctgtgaagaagccctttaggagccagggggcattggatgtgtgagctggccctggtctcatttctgattttaaaatggctctggctgtgatgtggagaagaggcagggtgagcctcatctgcagcaactgctaactgcccacatccccccagacttctcaaaaatttgccactcccctgctcaaagaccctcaaaagctgcccactgcttagaggacaggcctttgacctgaggtcctcacttgggcttcagggattctgtgaacccctaaaatggaatgcagaattaagtgtatgcaccttgccagaaacagggtttggggtttacattagatgctcagataggtcattgatccccaaaacactaaggtctctgacttctagacagatagtagttaagaaccaaagctttggagcttgagtgctggatttaagacccatgactgtcacagctgtgcaacctcagacaaatcactcagcctttctgtgccttggtttcctcatctgaaaatggagaaaataatattacctatttcatagattattaaatgcattcttgtatgtaaaagatgtaaaagaaaacttggcatgtggtaaactctgtgtgtttaagtgaatggtgaggctgcccagtataggagggcagagctttccaggtaaagactttctaggcctcctgggattatcaggccctctctgtgttggttgttgtcatacatgacagcagtattccttccaaagggatgccaagttgtggttgcacactgcacttagatccagttgattgacagcactgttcaagtcaactgtgtccttactgatttctgcctgctggatctgtcgattaccaaaagagttgaagtaatagtaataatactattattatccaataatagtattattcagaataatcacacagatttctctatttctccttgctgctctcagtttttgccttacatattttgaccctttgttgttagttgcctacacattaaggattgttatttacatcttcttggagaattaactcccgaatcattatatagtgccacttttttttaatttaaaaattttattttttattttttgagacaggatctttctctgttgcccaggctggagttcagtggcatgaacacagcccactgcagccttaacctcctgggctcaagtgatcctcctgcctcagccttcccagtagctggggctacaggcatgtgccacctctaataacttttctttaatttacaatagtattagtttgttctcatgctgctctaaggacatgcccaagactgggtaatttataaaggaaagaggtttgactcacagttctgcagggctggggaggcttcaggaaacatattatcatggcggaaagggaagcaaagacatcctttttcacatgacgtcaggaaggagacacgccaaggaaagggggggaaagccctttataaaaccatcagatctcaggagaactcactcactgtcacgaaaacagcatgggggaactgctcccgtaatctaatcacctcccatgaggtccttcccccaacacgtggggattacaattcatattacaattcaaggtgagatttgggtggggacacagagccaggccatatcacattattattaattagtgccttctctctcatttttttgattcgcctaggtagagatttatcaatttcattgatcttttcgaagaaccaatttttgatttcattgattttcctcgattaattttctatttttaatttcattgatgtatgctccaattttgtatttttttcttctgcttgttttagacatatattgcttttctgtttctagtactctagatggaaacagattattgattttagaactttctttctaagatatacattcaataatataaatttccctctaagcactgcttttgctacgtctcaccgattttgataggctatattttcatttttatttagttaaaaatatttttagtttctcttgtgatttcttctttgatccatatgttattcggaagtgtgtttttaaatctccaaatacttggaaattttctggctttcttcctgctagtgacttctaatttgaatccattgtggcctaagagcatatgttgtgtgatttctaatgttttcaatctcttaaggttgttttatgtcccagaatgtggtctgtcttggtgattattccacatgaacctgaggagaatgtatattctgctgttgtgagatgtaacagtctataaatggatacctctgttaatatgacctcgggtgactctttttttttttttaagttgctgcttcaccattggcttttggagattttgcagatgataaaatcctcaggacttttctgctgctcagtcagattttcttatcctacacttgcggaactgttgtttgaaattgacaagcaggatggccaactctgcccggctcattttatttcatcttattgggttcagcctgcagttttaggagatgagattgaaggaactggtatactggaatccacggtgggctcggaagcaaacaaacgtgggttctgattctagctctgaccttttaccagctgtgggacctggtggatgggttatttggcctctctggcctcagtttccttagctagacagttgggataacagcctacctttcaggattcttgtgtggattcggtgaggtaatacttctaaagcacctagcaaagtgtcaagtacaaggtaagtgctgggcaagcagggattttgttcacacaaagccttgactcaacataaggaggctgtttccaatggctctgcttaactgagacctgagagtgattgctccatctgtggtggcatatgggctgaaactgcctagcagcatgtcagaagtggggtgttcgcaggacatccttgtccaggttggcagttgagcttggcaggtgaccgctgagaccccttccaactccaccacttcaggaatctcttttttttttttctgagacagtcttgctctgtcacccaggctggagtgcagtggctcaatcttggctcactgcaagctccgcctcccgggttcaagccattctcctgcctcagcctcctgagtagctgggactacaggcacccgccaccacgcccagctaattttttgtatttttagtagagactgagtttcaccatgttagccaggatggtctcgatctcctgacctcatgatctgcctgcctcagcctcccgaagtgctgggattacaggcgtgagccaccgcgcccagccaggagtctcttgatttagaatgggaacttgaaatcatctcctcttgccgtgtacttgaacgcgataacctggcacttatctttttaattcttttactcacctgcatctttgttctttttgcctctgatgttcagggaagcctcttctcccactgcatctgtgatctctcctgaaatgtagcacccacacatccctgagaagctagctgcggcacgtgtgtgtgcacatgggcatgcgtaagtctgggcgtgccttccacaacagattaaggatatcaatcctgacgccatgctgggatgttttagtgaatcttgacagggttgttgactcccctgtttccctggaggactttaccatgaaagtcttctgctgtgtgactttggacaggcgtcttggcctctctgtttctctttattcataattgaggctaaagtgacacctttgccatctctgccacaggatactgtgggcatctgtgtagttgtgagagtaaaagttactaggacttgccaaggaaatcagtgtcaggaacccaccaccccattcccgcccctcctttacttgagactttacttttgtaactctcagcaaggaagacgagttcaacctttgagccgatacagagccttcccctagagagagctttgcagcctcatgctgagccagttaggggccccacatcatgtccaaaatgaatttaatcaacaaacagttactgaacacgtcctatgtgccaggcaccgttgtagatactggggactcggcagtgaatgggacagaccctggctgtcaggatgacagacaatttaaaaagggacagatgtcccacgctggcttgtcctttgcacccctggcacttcgtgaggagcagcggccagttcttgtatatagactggcccttggtgtacatttgtctgacgtttccaaatgatgtatgcatccttggcaggaacccaggaaaagacatgctgtgctcttctcaggaattgtttcagggaggtgatgttggggatttgtcccattcttgttggtgagtttagatcacttggtgaaggtggtgtctgccaggtttctgtattgtaacgttcattgatatgtattttcactcattagttaatatacaatacaagttttgcagggagttactttgaggctgtcatagtaaaatagagctttcccttcttccctgtttatttatttattagagatgaagtctgactctgttgcccaggctggagtgcggtggtgcgatcatagctcactgcagcctccaactcccaggctcaagccatcctcctgcctcagcctcccaagaagttgggattacaggcacacaccacactgcacccggcctcctccatttattgatctatgtcaatacagactcctggactttaactccaagggttacaagtggataccataatattcattttgatgctcaaaatgtgcctgttttgtccaggtcaggctagctgctgagtacttccttattgtctggcacaagaattccaagcctatctggtacttttccttctcctggaatcagctatttctctaaggcgctctggttcctttcagtacagaatagtatttagaagccaagacctggtcactaggaaggcatgataatcaaatgtgatgaatgatcttggattgcatcctggacgtataaagggcattattgaggcaattagcaaaacataaataggggccgtggatgagatagtggtgatgcatcactgttaatttcctgatttcgatggttgtattgtgattatgcagagaatgtccttgtttttctgacatgcacactgaggtattggggtaatttattcattatggagatatgtgagtgtgtttatgtgtctcatacacagatgtttcttaatgatggggttttgtccccataaactcatcataagttgaaaatattattaagtcagaaatgtacttcatactcctaacctactgaacgttatagcttaacttagctcggaacacttgcattagcctacagttgggcaaagtcatctaacacaaagcctgttttgttataaagtgttgaatagcttgtgtagtttatggaatactgagagtgaaaaacagaatggtcgtatgggtgctccaagtactgcttctactgaacacccattgattttgcaccatcataaattaggggaagagaggaaaagcacgcgtaagggaggaagggccgggtacagtggctcacgcctgtaatcccagcactttgggagaccaaggtgggcagatcgcttgaactcaggagttcgagaccagcctgggcaacataatgagaccccatctctatcaaaaatacaaacacttagccaggcctcatggtgcacatctgtggtcccagctactcaggaggctgaggtgggaagattgcttgagcccgggaggtggaggttgcagggagtcgaggttgtgccactacactctagcttgggtaacagagtaaactctgtctcaaaaaaaaaaaaaaaaaaaaggaaagggaggaagggaaggtccctccaggtggaggggacagagagagaaaaggcatggaggtgggactgagtttggtgtgtttaagaagtgcaggtggcagccgggctcagtggctcacgcctgtaatcccagcactttgggaggctgaggcaggcggatcacctggggtcgggagttcaagaccagcctgaccagcatggagaaaccctgtctctactaaaaatacaaaattagttgggtgtgtggcccatgcctgtaatcccagctactcagaaggctgaggcaggagaatcgcttgaacctgggaggcggaggttgcagtgagccgagatcgcgccattgcactccagcctgggcaacaagagtgaaactctgtctcaaaaaaataaaatgaaataaaataaaataaaatgttaaaaaacaaaaaagaagaagtgtaggtggcaggcgtaggatgcatgggtgctgggcccaggtggagagggaggtggagccctgctggggggagggccttcaagtgtgcccaagtcactcggagagcatccagcaggggcctagagctcctgaggtggccttaagttcttatgatgccccagacactgcacacagtgcttcagaggcaacacctccgttcctgtttgctgcaagcctgtgaggttagccttggaaccattcctgtgccacagaaacacagccagagcaccaaaagagaaatgctgttaatggcaaagaactactcactagcagaacaaagcctgggagctgcttttttgaataagatcccaggagagagacccaagcgggaagtgaaggatgggcacttgaaatgttgcagggaatataacccagcgcgtgctgttttctttttttctttttcttttttttttttttctctttttgcaacagggtctcactgtgtcgcccagcctagagtgcagtggtgccatctcggctcaccacaacctccacctcctaggctcaagcgattttcctgcctcagcctcctgagtagctgggactacaggcacgcgccactaccgcccggctaatttttttttgtatttttggtagagacaggtttcaccatgttggccaggctggttgtgaactcctaaagtgctgggattacagatgtgagccaccacgcccggccccagtacacactttttggagggtatttgacgttatgtaccaggagtgctggggaaaagtacacacttgactccgtgagattctgcttcttagcatttttttcttttcctttttttttttttttgagacagagtctcactctgtcacccaggctggagtgcaatggcatggtctcggctcgctgcaacctccgcctcctgggttcaagcaattctcccacctcggcctcccgtgtagctgggactacaggtgtgcgccaccacacttggctaatttttgtatttttagtagagacagggtttcactatgttggccaggctggtctcaaactcctgacctcgtgatccgcccacctcagcctcccaaactttctgttttcttttcttttcttttttttttatgacagggtcttgctggagtgcagtggtacggtcttggcttactgcagccttgacatcccaggctcaaacgatcttcccacctcaacctcccaagtagctaggattacaggcatgcaccaccgtgcctggctgattttgtttaatttttgtagagataaggtctcactctgttgcccaggctggtcttgaactcctgggctcaagccatcctccctccttagcctcccaaagtgctgggattacaggtgtgagccaccacacctggcttctgaacatttttttcttaatgataatagctggtgctatgggctgaatgtgttcccccaaattcatatgttgaaacttcatcccattgtagtcatattaagagatggggcctttggagaagtgattaagtcatgagggctctgtggttggtcatgaatggggttagtgccctcatgaacgggctgaaggaagtgtgtttgccccttctgtcatatgaagacacagcagcatggcatcgtccgtgaggaatgggctctcaccagatctggatctcctagtgtcgtgatcttggacttcccagtctccagaactatgcattagaaaatttatttatttattatttatttattttgagatggagtcttgctcttgtcgcccaggctggagtacaatgacgcgatctcgactcactgcaacctctgcctaccccagttcaagcgattctcctgcctcagcctccagagtagctggaattacaggtgcccgccaccacactcggctagtttttgtatttttagtacagacggggtttctccatgttggccaggctgatctcgaactcctgacctcaggtgatccacccaccttggctccccaaagtgctggaaatacaggtgtgagctactgtgtctggcctgaataataaaatttaaaacaatttttcaaaaattcaccatgaggtctcactatattccctaggctggtctcaaacccctggactccaagtgatccaccccaccttcccgagtagctgggactagagatgcacaccattgcacccaatagagcaatacgtttctgttctttgtaaattacctgctctaaggtatttttgttatagcagcctatatggactaagctgacttgtaacgttacttgagactttaaagtgttccggtcactgttggagggctctgtctgtgttagctcatttaatccccacaacacctcaatcagatggggctattcttagtcccactttatagataaggaaactgaggcatggaagcacagcttgctcaaggttcacatctagtcagtgacagagcaggtatttaaacctcaggaaataatcagagaaacatgtgtagagggttgtccaaggaaggccacatccagaagcatctcccaggacagttgttgtgtagctcaccctctggactttgtgggtctgggtgttgtttcatgattatagagagagctctgtgaacgtggaggacctgttgtcggcagagacacaaatggccagggcatggctgggcagccgcagtggctcaggcctgtaatcccagcacttcgagaagaccagaggggcagatcatgaggtcagaagttcaagaccagcctggccaacatggtgaaaccccgtctctactaaaaatacaaaaattagccaggtgtggtggtgggcacctgtaatcccagctactcgggaggctgaggcagaagaatcgcttgaacccgggaggtggaggttgcagtgagctgagattgcaccactgcactccagccttggagacagagcgagactctgtctcggaaaaacaaacaaacaagcaaacaaacaaacaaataaatggccagggcaggggagggttgcatattgaataagatgagctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgccagggtcctgcagtatgtcacctggccttatggagattacactgcagtgggaggggacagccaatgacaagtggccctgattatcagtaaattctaaagattgttagaaagtgatgggagccgggtgcagtggctcacacctgtaatcccagcacttcaggaggccgaggcaggaggatcgcttgagcccaggagttcgaggtcagcttgggcaacatagggagaccttgtctctacaaataataaaatattagccaggtgtggcagtgcacgcctgtagccccagctactcaggaggccgaggtgggaggatcccttgaactcaggaggtcaaggctgcagtgaactgtgatcgcgccactccactccagcctgcgtgagaaagtgagaccctgtcaaaaaaaaagagaaggtgatggggaaagaacacagaacagcataagagggggttggggaagctgggtggagtgggggggattgcagttgaaagtagggaagtcagggaaggcctcattgagctgacttggaggaagcgggaaccgtgcagatgtctggggaaggctcattcttggcagagaggccctgcactgagcctggcgggagggttgagcacaggagggaatgtggtggaggagagtgagcagcaggagggagcagtgaaggtcagcaaggtgacagagtggctgaatcaaaaaagaccttgcagtgtttgagcagaggatccatatcatccattatgttccaaaggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaaggtactttggggaggggcgggaatcaggagttagttttggacatgagataagcttggaatatttatttgctatctaagacagctccttaacatggtaagcccttatgcaagttgttgtcagctgagatgggcgtggcactgagcatgggagcatggaggcgcctgagtggtctcatgctcaggtggtttagcaaactcagtgtacatcctgccaattccagtcctgccatggccactgacaagctaggagggcgctgaaaggagaaggaccccgatgtctcctccagcccatccatctcctctctcccattggccaaacccaaccggaaactaaaggccaagggtacccggtgatgaagactgtggtatcagcctcctgagcacagagagggcagaaaggggtggagacaaagaggggcgcagatagtgggcaaatggggaagtggcacttcccctagctcgagggcagaggcttggtgtgatggaatggcactccttaaactgctacatattttccctttaatttggccaagaacaagttgtcaagtttgtgtgagataaaggtgcacttggttcgttcttgtctaatggcccccgcacccatgggtatttcttcagcttccacagtcatcccgacactagctgggaagctccagcagccctggtcctggccccagctctgtgggcgctggccctcaactttgcctgcactgtgcttttgtgctattccccttggtcctgtttgggtgcaagtccccctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtttgccccaagagtgagctgggtccaggtggtgcgctggagtgcaggatgctgagtatggtttgctgctgtttatatggtgttagaggggaggtcccatctccagggacatgttatgtaagatacagtggagcgcatggtgggagtgttggtccacgtggcacatggatacggctggaatactggactagaccagcagttctcacactttttggtctcaggaccctttttcacacttaaaaatgagtgaggacccaaagggctttggtgtaggtaacacatcattctatgtttacctaattagaacttgcaatgaagaaatggtgtaatttttaaaaaattaaaacaattaaaaattttttttcttactgaaatggaggtctcactgtgttgcccaggctgctctcaaactcctgggctccagtgatcctcctgcctccgcctcccaaagtgctgggattacaagcgtgagccgctgtatccggcccaaaatggagaaattttaagtcccaacaacatgcaagcccgcattcaacaaatcttcagatcaattacatgatcacaggtcatgtagcctctagaaaattccactgtacgccagtgagagagagtgaaaaggcaaataacgtccctgtattatgatgaaaagagttttacctggtgggcccagaccacactttgagaaccactggactagacccttgattgaggagtacggtgttgagagtggagtcctctgtgatggtggatggaccaggacacatggcataggagtcaggtggttccctgggctactccatggtgcacaggatgcttcgttacactggtgcccaggacataatcacgtacacaagacacacagttacggggcagactggggatatacggcacaccagcatgcagcgttcaccagtaaaggtggtattccatgattattctaaggtagatgggctgtgctttgtttccattggcttagtccagggattggcaaactatggcccgtgagccaaatccggcccactgcttgtttttgtaaataaagttttattggaacacactggctgctgtagttgtaacagaaactgcatggccctcctttatgttttttgtttgtttgtttgtttgtttgttttctttgagacagagtttcgctcttgttgcccaggctggagtgcagtggcacaatctcggctcactgcaacctctgcctcccgggttcaagcgattctcctgtctcagcctcccgagtagttgggattaatggtgcctgccaccacacccggctaatttttcgtatttttagtagagaccggttttcatcatgttggccaagctggtctcgaactcctgaactcaggtgatccacccgcctcagcgtcccaaagtgctgggattacaggcatgagccactgagcccggcctcctcctttatcttaattgaaataattcagaaatggaaagtcaaatactgcatgttctcacttataagtaagagttaaataatgtgtacacatgggcattattccatgtaccatggaataacagacattgaagacttgggagggtgggagaggggtgaaggaagagaagttacttaatgggcatagtgtacaccatttgggtgacggacccaccagaaccccagacttcaccactaggcagcatatccagtgagaacagatctgaggcttgccatcaaaattgcacttgtaaggccgggcactgtggtggctcgcggctgtaatcccagccctttgggaggccgaggtgggcagatcacttgaggtcaggagttcgagaccggcctggccaacatggtgaagctccatctctactaaaaatacaacaattaactgggtgtagtggcgcacacctgtaatcccagctactagggaggctgaggcgggagaattgcttgagcccaggaggtggaggttgcagtgagccgagatcacatcactgtactctagcctgggtgacagtgagactttgtctcaggaaaaaaaaacaaaaacaaaaaacaaaaaactcgtaccccctaaatttatacaaataaccaaaaaaaaaaaaaaaaaaggaaattgtgtggcctttgaagtccaaaatattaactatctggcctgttacagaaaaagtttgcagacccctggcctagcccgtgagatgtgggttggctgttaaggtggaacattggaattatcttacgatggccaaactgtgcgatgcagagcttatgttgttctaaattaattagtgccaccggttcttccctttcatgggctttcaggaacaagctaagtcccaggaccagggccggcagctaggcaggtgtgaggagcatccttggtgcatgtggtaagaggctgtggccagcaagagaggcaaccctagtcggctgccccagcacaccctggccgctcccaagcccccagatctgtcctcacatccgtgatcgggaagctggaagagtctgatgcggttcctggaggcatgtcccggacacagctgtggggcccagccagcctacaggtgaccagcctaacccagcccctgtgtctgcagagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctgcctgggctgagggtggggtgggcaggggaggaggtggggtcatagcaacagcaggaggaagccgcctgtattttcccaaatctgatgggattcctgcccctgcctgggcctcagtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacattccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatggtaaaaccccgtctctactaaaaatacaaaaaattagtctggcatggtggcaggcgcctgtaatcccagctacttgggaggctgaggtaggagaatggcttgaacccaggagaaggaggttgcagtgagcttagatcatgctactgccctccagcctgggcgacagagtgagattacgtctcaaaaaaataaaaataaataaaaataaaaagattttttaaaaggctgaagtttgggttactttggctcatacactttgccttcactgtagaaaggtggttagtaaagaccaggcgcggtggctcatgcctggaatcccagcactttgggagcccagcgcaggcagatcacttgagccctgggctattgaggctgcagtgagctgggattgtgccactgcactccagcctgggcaacagagtgggaccctgtctcaaaaaagaagaaaaaaagggtaattaataaacactaaagttctatgtagaattttagcaacattattgttattataatcttctttgctatggctctgaatctgtgtggtgctccagaagtatgctatggaggttttgtcgaccaaaaatctgggtggtggctgtggtttgtaggccggggctgggctgggtgatgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtgggtgaccagcagaggcccagtccctggagccaggagcctgggaggcaagccctggggctggatagcaaatcccaggagctagagacctggcttctcacctggctctgcactaggcaagtccctttgcttcctggccccccacccctcacatcagagaaggggagttatctctgcatgccgctcctcctctgtaaaggtagggctgtgggccacatctgtgtttcccagtttgggggacacaagtgatcgtaggtggcacattgacagctcacttgaataaccctattattgaagagaataatactgactcaagagacagtgacccgtgtcagttcccttttgaggccaacgggttaaggaggaagtccccatacagctgactcgtttactaattcctcttaatgaagagagcagaggccacaccccaggcttagactttcccaagaaaacaagatcagtttgttggttgttccccatggaagctggtcctgacattcccttcacagtagtgttggtggagtttttgttgttgtttgttttgagacagagtctcactctgtcacccagggtggaacacagtggcgtgatcttggctcactgcaacctccgcctcctgggttctagcgattctcctgcctcagcctcctgagcagccgggactacaggcacctgccaccgtgcccagctaatttttgtatatttagtagagatggggtttcactgcgttggccaggctggtctcaaactcctgacctcagatgatccactcgccttggcctcccaaagtgctgggattacaggtgtgagccaccgcacctggccagtggagttccttcttaagtacatgtattgacatctttaaaaagggcgagaggatttacaggaaactatcaggtcagtaatggcaggggccgtccacagtgggtggctgagtccccctatttttctgctggtgtgcagggaggtcatttcctgccacccatgtttccccaccctgaatccaccttcctcacattcccattggagggacaatctctggacatatgggacctggggtcccacagggctgcaatccaatgcctgctgtgccactcgccagctgtgtgatgttgggcatatcccataacctctttgtgcctcagtttcctcatctgtaacacaggagtgacaagagcacccgcccacagggctatgacagtacaaggtgtgtgatacagatgagctcccctgtttggcccacatgtgtcctaaaagccatgtgccctttctcttgagtgccccaggccacagagatccccatctgcccgctgtcccacacactggtctgtcatttgttccttgaggtttgtgagggccggctctgtgcatcccaggggcccaggctgggcctggttggctctcagggagcaggcacccgccaccttaagctcccatgctggtgtctgtcactgcttcctctcaatctggccaagccaggggtgtcgatttatatctctcaggtctggtttcccctttggcactgggccaggtatggggaaagagcaggaatggggcagttggctcacacagcagaggctcagaaagcggggggcatggggggaaggagtgcacagatgctagagagtggggcaagttttgtttggtcaataaatctccttctcatgccccaggcctgtgcaagacctacagagagtcccaaggatgggctggggggaagagaaaggtaccaccttcagagtccaaagatatgttatttaatattttcatatttctagatctgccttcaggcatggctggatccagcttctaggaacctgtccagctctgcgccctgctttattctgtactggcttcgtttttaggcaggctcttccctcatgtagtggcagatatgcctactagttgctccaggcctacatcccaaagccacagtgggaaaagggttttttttcttgacggttctaataagagtcctaaggctgctgctcagtggcctggcttcgatgctgtgccagcctctgaaccaatcactggctgtgggtggagagagggtgctggtggagggccctgcttgtccagggaggagtcacatacctgcctctagggctgcaggtgggctcagctccatccaaaccagatgaactgaaaataaggcaggagtggcttccccaggggaaactggggaagaggaagcaggactgtgctggctaaaatgccagccaggtttaagacgtggcaccagatgccagtcatgggattggattggtcagcatgcctgggctatggcttaggggtatgttggtgctcagggatgccacaggcctccagataccaggtctgaggcagaagaatgaagtccagcttctcttgtgggtggaacagtggcaactgagataccccatctctcccttcccaagaacagagctgaacataaagaatttagtgattggccagagcttggccacatgctcccctctgatgaatgataggccaggtgatgggattggcacaattggcttagactaatgagggttggccctggagttgcaggcagtggagttctgtcctaagcagtgggcacctaaacccgatggcataaaagctgggcgggtgtccacctgcatctgccacagcactataggcaccaactgtggctcatactgagtgggataaattccagaaagaaacattaggaacttactatagaattttggggctagagctactcattcattcccctagataatttctaggcaaggttccatagtggagggggagttttggcttgggcattgaaggatgcataggagttttctagatggggaaagaagggaacggtagaccaggcagagggaactgcatgataaaaggtttatgggtgtgaaaattcatggaatgtttgaggattatggggttgggggatgtgggaatatgtgtagcgataaagcaccaaacaaagccaaaagtttagttagagccctgaatgcctgcctcataatggtttccatattttatatgcctactatgtgccaggcacattgctcagggtcacacagctggaaatggcagggctgagtttttgttgttgttgttgttgttgagacagagtctcactctatcacccaggctggaatgcaggggcgtgatcatggctcactgcatccttgacttcctgggatcaggtgattctcccacctctgcctcccaggtagctgggactacaggcacaggccaccacgccaggctaattttttgtatttttagtagcgacagggtctcgccatgttgtccgggctggtctggatctcctggcttcaagtgatccccctggctcagcctcccaaggtgctgggattacaggcttgagccaccgcatccagcccagatctgagatttgcacccagtatttgaactcccaagcctgtgctctttttcctcccatggacatttctctcagagatggtctcccaaacacctgtccttcttgttaaaaaacagacaaaccgcaagtagttctttggaagctcagatttctcttttgtttcttagtaaaacatttcccagttcccagctcccttccagggtgtaagatttcttcggtaacttacatctagctgttgcttcttgtttgctcatgtttagaaagaaagacaaaagagagtgagaattttctctcccttccccagtctccccacaactcacaccccaccctcagctccctctgtaataggaaaatctctgaactctctgtagttgctccagcaatcttttggaactttgcttctttcttgtgaaaaaacctccccttggctcactttgcaccaggtttccccaaatgtgcttccaaccacaagcagaaatggagctgccagtaaccaggaagaaactgccgggggctgaggaagaggagagggaggtgcatagccctggatctcgcagggagaggggtgacaggatgagaactcaggttgctcacttgccatcagggtcagtcatgaatatagcgttcatgtatcactttttaaagcttttttggagggtaaaagtaatagttacacaaaataaaaatacaaatggtacaaaaggacttagaatggaaacatgtttctctcccgactccagcctcctgtttttcttcccagagactgaccactgctgtctgtctcttgccagaagggaaagggaggcaaggttagggcaggcagagggcatgtgcatcctttagagagagcttatgtctatacaagcaaatgtgtgtgttcagtcatcgctgtcttagttttctattgctgcataataatggtactaccagcttcacagctttaaacaacacccatttattatctcatagtttctgtggttgggagtctggacatagcttagccaggttctctgctttagagtctcgtgaggctataatcaaggtgtgggatggggctgcagtttcatctgaggctcaattggggaagggtcacttctaagctcatacaatattggtgacattcagtccctggcaggctgttgaactgagagcctcagtttcgtgctggctgttggttgtagttaaccctgaattccttcccatgtgccctttgcaaagccatcaaggcagagagacttgcctagcaagtaggatattacagtcttctgtaatataatcacatccatgaaatcctctatatatcccatcacctttaccatattctgtgggttagaaacaagtagcaggtcctgcccacactcgagaagaccagatgacacaaagatgtgattcaaagtggggatcatcggggccatcttaggtttgtctgcagtgatcactgtgccatctctctctctctctttttttttttttttttttccgagacgaagtcgtcactctgtcacccaggctggagtgcagtggcatgatctcagcttaccacaatctctgcctcccaggttcaaatgattcttctgcctcagcctcctgagtagctgggattacaggtgcccgccaccacacccagctaatttttgtatttttagtagagacagagtttcaccatgttggccaggctggtcttgaactcctcacctcaagtgatccacccacttcggcctcccaaagtgctgggattacaggcatgagccaccatgcccagccccatctctctttaaaaaacaaacaaacaaacaaaaaacataaaaagaagcagagaacacatacacatctgcatcttcccttgtttacttaacaatagatcttggaagtcacttctcagtagaggctaggttgggcagagcattggattctaggccagtgagtttggacttgaccatggagacactaggaagcccatgaaggacagagagagatgcctcgaccctgccagtcctttagaaagatcacccagtgctttttgtataccaaaccctatttgaaatacttacgtatattaacccatttccttatcaccacaaccctgcgggaagggagataggcacttttattatcttcattttgcagatgaggacattgaggtccagagaggttatgtcacttacttaaggtcacacagccaggaagtggtagtagggactcttacccttgttttacagatgagattgaattatctcacgaaaactcagaaaggttaaacaacttgcctaagtaacatacagctaattagtcgaggagcctgacgcatgttgctgtagcctggtcacagttacagaggtggcaagcaatggcctgaacaggacgaacaaccaaatacccaggctggtggctcttaaacatggtggggtcagctaacgacagcaaccagggtgggcactggtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggcttttatatcataggatgcctctaatggcaatcctgccattagatacacctgccgtggtgtatctgccaggtaggcaggctaggctgcagtaacaaacaagcccacaatttccatggcttaacactatgggaatatatttcttgctcacgtaacaagctaacgtgaatgttgctggtttgtaggtggtttccctccctgtagaaatctggggagtgaggttctttccatcttgtggtgccgtcattctccaggacaaagattcttacctacttttgtgtcctggtttcctttggcagcctggtgaagcctatggacctcatttcagaatatttttaaatacataaaatcccagcctgggcaatatagtgaaacccccatctgtacaaaaattagccaggcatggtggcatgcacctgtagtcccaggtactgggaaggctgaggtgggaggatcacttgagcccaggagtttgaggctgcagtgagccgtgatcgtaccactttactcccacctgggtgacagagcaagagcccatctctaaaaataaataaatacaatgaaataaaataaaataaatagaactacagaggaaactaattgtattgaaatgcagttataaaacatttaaacacatttttaatctagagatatatgtgcttctttattaagatctataaataataagttctaggggtagctcgcataaatactgtaatttcaaagtagataagcataaataatactttatgatactgaaattgtgatgtgatatgagaatagctgtgagttttgttttgctggggacaggatcagtgatgctgtcattactggggtctcttccctccattctttttttaaaattgtattttattttatttttaaaattttaaaataaatagagacagggtatcactatgttgcccaggctgcttttgacctcctgggctccagtgatcttcccatcttggcttcccaaagtgctgggattacaagtgggagccagtgttcctggccccttcctccattcttaatggaaggagatgctaggtgtgagaggttagggaaagtaaagatgtaatttctttcccatccaagttctcagacccctgaattctacctgcagccatgttggtccatcaaccccaagtgaagaatccctgctctagggccccaccattgtctgtatccagccagcagaagaggcgtgattatggagatcacatctgcttcttgaaagcagacagcccggaagtgggccgcatcacttcctctcaaattctattggtgaaaatggtcacatgactacacatagccacaaaggaggctgggaactttctcacttggaacctacatcccagaaacaactcttttcagtgaggtatcccacaggtctttcgcagtagaaatattgattatctcacataaaatgaagtcttacaaatggacctactgggttttgtacagcagccaagtgatatctcttcccttctgctgtcttcccttctgccatccttcacatggtggcattgtatccttagacttgccacccatgccctcaggttggccgttgcacactgtcttacataaagcaggaaggaaaggaaaggctgctacgagagagtgtaccttgtgcatctcttttttaatcaggaagcaaacatctttctagaagcttccctagcaaaattccccttacatctcattggccaagactgttacatgttacatggttactgttattacttgctcattgcaaggaagactgggaactcaaatgcctggaaaaaggaacaggataatcgtgattggctcaagccttagggtgggcatggctccctgacaagggagagaggaaaaagctgttgagtgaagaagactgcttcagtttccccatctgtataatgggaggagtaagggctgtcgtaaaaactcaatgaaagaagattcttcaacgtggtaggtgcagtggcagctggcagtaccctgaccctgccaccgcacagccctctcagcattgctcatcctgcactgtggatatcagttgagccacgtgtctcctgccctgggctgtgagctccataggcagggtctccatggctgtatctccagaacccagcacagaaccaggtgcttgggaaagttttgaattgattctcatctgccattggcatggggaagggaactagcttgtatgaaacagataacaatgtatgggaccctcattcattatttcagcaaatatttgctgagttcctcctacatggctagccctgtgctagacactggggaatcggcgatgaacaaagcagatagaaatccccactcttgtggagctgacattctggagggagagacaaaaagcaaacatataaagaaagaaagaaatcacatggatctggatgacagtgagtgctgggaagaaaataaaagcagaggaaggggatggagcgatgggcagggggcaacggtagggagggtgtcggggaaaactttttggagaatgtgacgatgaaagtgaacaaggagaagtcaaccgtgttgagatgatggcagctaatgatgtggacaggccactctgttctgagtgcattatctattgattcatcatgtcatcctcgcaacagccctgcacgatcaattctgtcattaaccccatagtacagatgaggatgcggaggcacagagaagataagggacttgtcctgtgtcacacagcaaggagccatccggctcctaagttggtgcatttgacttctgtgcttccggaaagaaagagcagcaagtttaagatctggaggtggcactgagctttggaggagcagggggcaatgaggtggccggtgtgacgaggactcaatgtgcaagagggagagtggtggggagatgaggtggaggggtggtcggcggtcagatcgtggagggtctcggacgagggtcctgaccctgggtctccagtcctgggaagtggagcccaggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtggcagggacttgcccctctagtctgcccctttgcagtcctctcagtcaataatacgcatttactgagcagctactacacaccttgagagtagagctgagaacatatcgacaaggaccccacttttttctttttttttttttttttttttttttgagacggagtctcactctgtcacccaggctggagtatagtggcacaatcttgcctaacagtaacctccgcctcccgggttcaagcaattcttctgcctcagcctccagagtagctgggattacaggcgcatgccactatgcccggctaattttttgtatttttggtagagatggggtttcaccatgttggtcaggctggtctcgaactcctgacctcatgatctgcctgcctcagcctcccaaagtgctgggattacaggtgtgagccactgcacccaaccaggactccacatttctaaaaccggcatcctactggggagactgaaaatacatatcaatcacaaacaggtggttttccatagtgacccactctctgaatgcactagaccagggtgcaggccagagatcttctggggtgctttttgcaagggggaccaggataaggctctccaaggagggaaaatttgaggggggccctgactggggagaatgagctggccagggataagcaagatggagtcatcccacatccccttacaacgctgggtgcctgggcaactgggggcatctgggggcatgtggtaggagccagaggaatttgcgacgattgccctgatggagtcaggagacctgggtttgaatcctggccttggagcttggtagctggcggccgacaagttgctgaaacccctgagcctggggttcctgctttgcagagtgacagtgatggtgagaacatatttcatcagccagaagaggccaaatcacagtaaaggctgagggaggagatgagtggcgagtggctgggaggtggtggaaggagcctcgtttccagagagctcttgccagcccttggaatcatggtgtctcagagcctcagtcctcccatctctgaaatgggactagcaagctcaacctcactaagtcaggattagaggtggctaaggattattaacatgattgatgaaagtgcccactcttggcccagcacacactaggtaggcagggaatgcaaattcccctccatatcttgtcactgatgcctccgagcaaccttggactgatcgccttgctctgagcctcagtttccccatcacctgtacctcttcccactccccatcactatatcccagcatgccagcctctttgctgttctttgtctttggtttcttgttttgttctgttttttagacagggtctcactctgttagccaggctgaagtgcagtggcgcggttacggctcactgcagcctccaattcctgggctaaagagatcctcccatttcaacttccagagcagctgggacaacaggcgcttgccaccacacctggctaattttcttattttaatttaatttaattttattttttgggacagagtggagtctcaaaaaccaagctggagtgcagtggtgcgatctcgactcactgcaatctctgcctcccgggttcaagcgattctcctgccttagcctcccgactagctgggattacaggcgtgtgccacgacacccagctaatttttgtatttttagtagagatggggtttcaccatgttggccaggatggtcttgaactcctgacctcaagtgatccacccacctcgttctcccaaggtgctgggtacaggcatgagccactgtgcctggccaattttcttacattttgtagagactggctgtcacttatgtagcccaggctgatcttgaacttctacccctttatctttattcatggcacttattaccatgaatgaatgacctcatataagcatttctttcgtttttttttttttttctttgagatggagtctcatgttgtcccccaggctggagtgcagtggcgcgatctcagctcactgcaacctccgccttccgggttcaagcgattctcctgcctcagcctcctgagtagctgggattgcaggcgcctgccaccatgcctggctaagttttgcatttttagtagagacggtgtttcaccatattggccaggctggtctcgaacttctgacctcaggtgatacacctgccttggcctcccaaagtgctgggattacaggcgtgagccaccatgcctggcctcatataagcatttctgtctccatttatcatccatctttccctcttgaaggtcagtttcaccaaggcaggcatctttgtctcgttcactgttgtggcctcagggccaggcacagtgagtcaaacatagaaggtgctcaataaatatgtgtttatttattgaaaccatgggcagaggctaattcagaagcggtctgaggaccttacctcccagtgatgatgcaccatggccccaggcaggccaggaagagagaagggttgtgtttctccgtaggtcccccagcttcccaggccatcccaggccattccctggtcatttgccctcagctgctctgaaaaagggattgttgaggggaacctagaatcctctctctgcagtttgagtctttcctaatcccctggggtctcattcccactgaggacataggtggcctcctcaggaactctgtgctgggtaacagaatgcgggagtgtgaacctggctctgccacctaccagctgtcactccacctccttgggcctcactctcctcatctgtagaatagggttagcaatagaatccatgtcaccaggttagaatgatgagtcagtggtttgacctccagaaactaatcagcctgatctctgatgccaaataagtattggtgataacgaccacttttatgggaggagcgttcacctgtcaataattcagagatcaacaccttttccttttgtttttcaggattaagaaagaatggtgggatcagattcccaacccagcccgcagccgcctcgtggctataataatccaggatgctcaggtaggagtaggcgtggatgaggacatgtgggactgtgtacatgaagaagtgtggttcagaacacctgggctgttaaggaccttcactggcttctggaatggcaaatagacagtcaggagggttgcaggggagacagagacagaagccgaatgaggtcattagcagaccagaggctttcccgcccttccccttggcaatcccagcctggggtgggcttctctggggttggtttcctgtttttttccctccccttgggagaatgacccttgggtcatcatcactgtgtcattccctggggaggtgccagtaccagggctagaggccagaaggagtggaggaaggagagggtgacaggctttctgtgtcttcttcttaagcataggaaactgcccccgaagcactagcaaatcccttccgggttctcattggcctgaaatgtatcccacccctaagccaggggtggagtcagcttccccaaggcgatggtcctgtgggtgagtgggtggggtttgcctgagcaagatgagagttctctaggtaggagaaagggggattataggtcctgtctagaagagaaggtctgagggtccttgcttttccagggactctggaatctagtggtgttggctttgaatcctgactctgccactcactggcagtgtggacttgagcaagttgcttaattctctgagcctcagtttcctcttgtgggttataacagtgtttacctggtaggacagatattggaatttattgagacaatacatataaagtgcatattccagcctcttgcaaataccaagtgccatttatgtatcagttagtgtttgctgtgtaacaaacgaccccgaaatgtagagggttacaacaactttatttagcttatgcttctgcaggctggcatttggggctgggctcagcagtgagggtggcgggggaggctgggctgggctgggctgggcagatctgaattgagctgacccgtccccgtagcctccctccgtgtctgacagttggctttttttttttttttctttttctgagacggagttttgctcttattgcccaggagtgcaatggcgtgatcttggctcactgcaacctctgcttcctgggttcaagcaattttcttgcctcagcctcccaagtagctgggattacaggcatgtgccaccacgccaggctaattttgtatttttaatagagatggggtttcttcatgtcggtcaggctggtctggaactcctaatatcaggtgatccacccacttcagcctcccaaagtgctgggattacaggcgtgagccactgcacccagcctagttggctgacttttacctgggacagtgcaggtgcctgagccatgtgcctctcactctccagcaggccggcccaggcttgtttacagagtggctcagttttcaagggtgggaagtcccaaggcttcttgaggcctaggcgcagcactggcatgatatcacttccatcacattctatgggcccaagcaagtcccagggccagtgtagattcaagggatgggaggagattcagagcactcctctgtggccacttttgccatcgaccacagtccctgtaaatattaggacaatgtaattaattcccaggaatctgaagctcagaaagcgtaagtgacctgttggacttctgatctgtgtgatgtcgaggcttgtaccccttcctgagcattgccgtactccaggccgggctgcaaggccactctgctctttcattggctgtctctgtattttaggggtcacagtgggagaagcggtcccgaggccaggaaccagccaagtgcccgtatgtatctgaacttaggtcacagcctgcatgcattgggaaggtgatagaattggagaggcaagcccctagctccatgtctgccttctcttccctgcattcggtaattgccctgtgacattagccttcaagggacggcaggaggaggggtgttctggaaacgtggactgctggccaagccccctgagtttcactggtgtgtcaggtacatggtgataccccttgggagtgctgttatagttaacaaccagagcagccgtgcctgttgttaaaatcttgacctaattgtatacttgtcggcaaatagccactatcctgaacactcccctccttttttttaatatacaggatctcactctgtggcccaggctggtgtgcagtggtgcgatcatagctcactgcaccttcaaactcctgagctcaagtgatcctcccatcttagcctcccgagtagctgatactacagatgtgcattaccacgcctggctattttaaaaggtttttgcctgtaattccagctactcaggaggctgaggcatgagaatcacttgaacccgggaggcagaggttgcagtgagcgcagattgtgccactgcactccagcctgggcgacagagtgagactcttgtctcaaaaaaaataataccaaaaaaagtttttgtaaagacaagctctcgctgtgttgccccgccactgtggcctccttagcttcttccctggggcctgctggacctttccatactccagaaactaaagggggtccaggaccctgcttcaaccctaggatcccgcatcttttttttttttttttttttttggacgcagggtcttgctgtgtccctcaggctggagtgcagtgattcactgcagcctcaaactcgtgggctcaagtgattctctagcctcagccttctaagtagctgggactacagtcatacaccaacatgcccagctaattttccttttttttaattcttgtagagatgtttgagacggcttgggctctgttgcccaggctgttctcaaactcctgagctcaagcgatcctccctcctcagcctcctaaagtgctgggattacaggcgtgagccaccgcacccggcttccatatcctttctaattggtcatggcttgggataatggtgttgcttttaattatcatcatccataaagactttttcttactcaacagatctgagcttgtatttggtgcccaggacatgtgctgggttcccgaaatcccaaagacacagaccctaccctcagggatttctcattctagcaacatagactgatcaattactgattataacgttagaaggcatgtctgaagtagacagccatcaggacatggtgatttcaggctgggctttgaagaatgaataggagtttttcaagtgtcgaaactgaaccctgaccaacctttgcttttgcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttattttttatgtattaaccaaacatacctccagacactgctgtgagtgctgtgtctctgttaactcctggaattcacccatccagaggaaccaggatgcaagaggttaagaaacttgccatctgggtttgggttccccatacaaggattcaaatagttgatttaggaagtaatcccgggaaaccctgctaaggtagtggggaactgaggcagggaaggacacaaaccaagaaagtgttacctgaaaggggtccagatgcagaccccaaaagagggttcttgaatctcatgcaagaaagaattcagagcgagtccatagagtcagtgaaagcaagttaatgaggaaagtaaaggaataaaagaatggctactccgtagacagagcagccctgagggttgctggctgcctatttttatggttattgattaattatattccaaacaaggggtggattattatgcctcccttttagaccatatagggtaacttcctgatgttgccatggcatttgtaaactgtcatggcgctgttgggagtgtagcagtgaggacaaccagaggtcactcttgttgccatcttggttttggtgggttagagccatcttctttactgcaacctgttttatcagcaaggtctttatgacttgtatcggtgacgacctcctgtctcattctatgactaagaatgccctaacctcccaggaatgcagcccagtaagtctcagcctcattttacccagcccctcttcaa

SEQ ID NO: 3 - DNA, 3597 bp, Homo sapiens
ggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctc

SEQ ID NO: 4 - DNA, 2462 bp, Mus musculus
gcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgccgtggactgcagttctcagctccgcctgcactacaggctgatgttcttcgagttctctgaaaacctcatatgcatcccgaggaacagcgccagcaccgtgtgtgtgtgccacatggaaatgaataggccggtccagtcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcattgtctataatgtgacctacaaggaacccaggctgagcttcccgatcaacatcctgacgtcaggggtctactatacggcgcgtgtgagggtcagatcccagatactcactggcacctggagtgagtggagtcctagcatcacgtggtacaaccacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccctgcttgctgaagcacagagtaaagaagaagacagacttcccgaaggctgccccaaccaagtctccccagagtcctggaaaggcgggctggtgtcccatggaggtcagcaggaccgtcctctggccagagaatgttagtgtcagtgtggtgcgctgtatggagctgtttgaggccccagtacagagtgtggaggaggaagaagatgagatggtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcagggagtgggcaggcttctgtatcctgggcctgcctccccatggggcccagtgaggaggccacatgccaggtcacagagcagccttcacacccagaccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacggtgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctatgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtg

SEQ ID NO: 5 - DNA, 6649 bp, Mus musculus
  modified_base (1081)..(1180): a, c, g or t
gaattcatgctgctttctcggcctagttctgctgagcttctctgagcacctgagccaagcagggctggtcccaagcaccatcacaggccagcctgtttctgtcacatatcgatgtatctcacatagtcccctctcccctgggcaagggtcatacaatccagtcacatgaagacaagtggccagccaggagggatcaaaagatggcttgcagactcagaagacagcaagatgtggtggcttgtatctgcaatcctacttgggaggctgagacaggattactgtgacttcaagttcagactgtgctacaatatgacagccaaagctgtatagcaagatcctgtcacaagaagcaaaaaactaagccaacccaatccataatgctcaaaacaggatggcaagcccatgggatggcttagctagcaaaggcaaggctggtcacagttgcgctgctgtgggcgagagaacaacttcgtgatgtcagttctttcttttcacttttgcgtggtttctagggatagatcttgcactctcaggcttgcgggccatctcatcaacccttaagcaagtttcctaacagatccatgcctcagcttcttcatctgaaaagggatagttgcgcatgactccctcttggtagtagcagcaaggttcctgcctgaacctagcaggagacactcgctcttaattgatgagttgttttgctttctggcacagacgctgagtaaacattgggaaaatgagcaacagccgcaaacacaagccaagctggtctgtagggaaagggcaaagattcgagtcccgcagtcgtgggacttaaaagctggagctctggacagctgcggacgcgtggagtggggcatgcaaataagccagcgatcaggcaaagtccggaggactgcgggtgaaaatgagctgggtgaggggcggggcatgtaaatgaccgtgcacggggtggggcgtcgcatgtaaataagggacaccaggcggcgtaggaagcggggcgaggtccttctcgcaggaaagccccgcgcggcgcgtggagcctgaactcgcaggtaggaacgggcggtgggaaagcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatggctcattttatttcattgttaggttctggctggacttctcgaagctgaggagaagcagagggacctggcttctgattttggatctgcgtgcttgctggttctggggcctgctggtcttgttcctgtaacctaggactcggggcttgcacatgctttttttttgaagttgctggagagggagcccaggaccttgtgcgggtgagccaggtatcctgacactgcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatctgcccactcttctttctgcaggcaccttttgtgtccccaatggggcggctttgcaccaagttcctgacctctgtgggctgtctgattttgctgttggtgactggatctggtaagtcactcattcattcatcaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatctggccggttctgtctctgcagggagcatcaaggtcctgggtgagcccacctgcttctctgactacatccgcacttccacgtgtgagtggttcctggatagcgccgtggactgcagttctcagctctgcctacactacaggctgatgttcttcgagttctctgagtaagtggggtagggaggcagcaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtaacagagtttctatgtctgcagaaacctcatatgcatcccgaggaacagcgccagcaccgtgtgtgtgtgccacatggaaatgaataggccggtccagtcagacagataccagatggaactgtgggctgagcacagacagctgtggcagggctccttcagccccagtggtaatggtaagacctgtctgggtgctccagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacccaggtgcccttcctccccagtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaagtgagtgggcgccacggatccctgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcatatgtccctgtcttccttcagttcattgtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaaccgtgagtatcagggtcgtaggctgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtacagcgcacattgtttttttcagcaagtaatgaaaatctgtgactgagtgaccttgggggctgcggtggtgaggagagctcacgggaatcctggagcagtgtagctggcgtgtcaaaagcagaaacgcaggagatgggtcagtgcgccgaggcccggagtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcatgactgtttttctccttgcagacttccagctgcccctgatacagcgccttccactgggggtcaccatctcctgcctctgcatcccgttgttttgcctgttctgttacttcagcattaccaagtgagttcctgctttggctggtgtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaacctacttcctttccaccctcaggattaagaagatatggtgggaccagattcccaccccagcacgcagtcccttggtggccatcatcattcaggatgcacaggtaagagggtacaggcgttgcatagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccactgattggctgtctattttaggtgcccctctgggataagcagacccgaagccaggagtcaaccaagtacccgtatgtatctgaacttgaacttgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttaaccctcagctttacctctgcaggcactggaaaacttgtctagacaagctgctgccctgcttgctgaagcacagagtaaagaagaagacagacttcccgaaggctgccccaaccaagtctccccagagtcctggaaaggcgggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcagggagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggcccagcgggcacgataactggcagtcaaggcgatacctcgagggaactgtggggctggtcctggttggtggtcaggtggtagggatagcagatggcagactttggtgagtgagtgagtctgactgtgttctggaagatgggaccaggctcagcactgtctgctcacgtccccactgttgcgacacctagtctgtttgcaaggaggacaggacaggtcacatggagctttatgtcaataaagtctttatcttgtcaggtttcctttactatacacacgccgagcccacgtgcacgacagttgaaatgtgcaggcagggggttggggaagtggggagacaaggccccagcagttggtttaagggaatgacttgggaatggggcagagctgtggctactccatctctcatccttaccctcctgctaccacagcttgtgcccatgtgtgcctgctcaggggaggggtcctcctctgtccctctcctttaggcagagtgctagaggtgttggatgctccttagcacgcagaggccgtgacactggctgc

SEQ ID NO: 6 - DNA, 496 bp, Mus musculus
tcgagagcggccttctcgcaggaaagccccgcgcggcgcgtggagcctgaactcgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgccgtggactgcagttctcagctccgcctgcactacaggctgatgttcttcgagttctctgaaaacctcatatgcatcccgaggaacagcgccagcaccgtgtgtgtgtgccacatggaaatgaataggccggtccagtcagacagataccagatggaactgtgggctgagcacagacagctgtggcagggctccttcagccccagtggtaatgtgaagcccctagctccagacaacctcacactccacaccaatgtgtccgacgaatgg

SEQ ID NO: 7 - DNA, 2043 bp, Mus musculus
ctcgcaggttctggctggacttctcgaagctgaggagaagcagagggacctggcttctgattttggatctgcgtgcttgctggttctggcgcctgctggtcttgttcctgtaacccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggcccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtaaaaaaaaaaaaaaa

SEQ ID NO: 8 - DNA, 3583 bp, Mus musculus
cgcaggaaagccccgcgcggcgcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaaccacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggccccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtc

SEQ ID NO: 9 - DNA, 3697 bp, Mus musculus
cgcaggaaagccccgcgcggcgcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaacccaagtaatgaaaatctgtgactgagtgaccttgggggctgcggtggtgaggagagctcacgggaatcctggagcagtgtagctggcgtgtcaaaagcagaaacgcaggagatggacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggccccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtc

SEQ ID NO: 10 - DNA, 3565 bp, Mus musculus
gcgcggcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaaccacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggcccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgt

SEQ ID NO: 11 - DNA, 19 bp, Artificial Sequence
  Description of Artificial Sequence Synthetic primer
aatggtcccaccaattgca

SEQ ID NO: 12 - DNA, 23 bp, Artificial Sequence
  Description of Artificial Sequence Synthetic primer
ctccgttgttctcagggatacac

SEQ ID NO: 13 - DNA, 21 bp, Artificial Sequence
  Description of Artificial Sequence Synthetic probe
tttttctgctctccgaagccc

SEQ ID NO: 14 - DNA, 19 bp, Artificial Sequence
  Description of Artificial Sequence Synthetic primer
cctggagcaacccgtatcc

SEQ ID NO: 15 - DNA, 18 bp, Artificial Sequence
  Description of Artificial Sequence Synthetic primer
tgccgggtcgttttcact

SEQ ID NO: 16 - DNA, 39 bp, Artificial Sequence
  Description of Artificial Sequence Synthetic probe
ttacctgtataatcatctcacctatgcagtcaacatttg

SEQ ID NO: 17 - DNA, 21 bp, Artificial Sequence
  Description of Artificial Sequence Synthetic primer
tcccattttgtccaccgaata

SEQ ID NO: 18 - DNA, 20 bp, Artificial Sequence
  Description of Artificial Sequence Synthetic primer
gtttctaggcccagcttcca

SEQ ID NO: 19 - DNA, 25 bp, Artificial Sequence
  Description of Artificial Sequence Synthetic probe
tgtcactcaaggctctcagcggtcc

SEQ ID NO: 20 - RNA, 19 nt, Artificial Sequence
  Description of Artificial Sequence Synthetic antisense compound oligonucleotide
cgagaggcggacgggaccg

SEQ ID NO: 21 - DNA, 21 bp, Artificial Sequence
  Description of Combined DNA/RNA molecule Synthetic antisense compound oligonucleotide
cgagaggcggacgggaccgtt

SEQ ID NO: 22 - DNA, 21 bp, Artificial Sequence
  Description of Combined DNA/RNA molecule Synthetic antisense compound oligonucleotide
cggucccguccgccucucgtt

SEQ ID NO: 23 - RNA, 19 nt, Artificial Sequence
  Description of Artificial Sequence Synthetic antisense compound oligonucleotide
cggucccguccgccucucg

SEQ ID NO: 24 - DNA, 20 bp, Artificial Sequence
  Description of Artificial Sequence Synthetic antisense compound oligonucleotide
ccgctgttctcaggtgacat

SEQ ID NO: 25 - DNA, 20 bp, Artificial Sequence
  Description of Artificial Sequence Synthetic antisense compound oligonucleotide
tggaaaggcttatacccctc

(19) United States
(12) Patent Application Publication
(10) Pub. No.: US 2010/0056606 A1
(43) Pub. Date: Mar. 4, 2010

(54) Combination therapy using budesonide and antisense oligonucleotide targeted to IL4-receptor alpha

(75) Inventors: James G. Karras, San Marcos, CA (US); Susan Gregory, San Diego, CA (US)

Correspondence Address: SWANSON & BRATSCHUN, L.L.C., 8210 SOUTHPARK TERRACE, LITTLETON, CO, 80120, US

(73) Assignee: ISIS Pharmaceuticals, Inc., Carlsbad, CA (US)

(21) Appl. No.: 12/066,633

(22) PCT Filed: Oct. 3, 2006
(86) PCT No.: PCT/US06/39168

§ 371 Date: Mar. 20, 2008

Related U.S. Application Data
(60) Provisional application No. 60/723,426, filed on Oct. 3, 2005.

(51) Int. Cl. A61K 31/7088

(52) U.S. Cl. 514/44 A

(57) ABSTRACT

Provided herein is a method for reducing the amount of steroid required for the prevention, amelioration and/or treatment of pulmonary inflammation and/or airway hyperresponsiveness, comprising administration of the steroid and an oligonucleotide targeted to IL-4R alpha. Also described is a method for the prevention, amelioration and/or treatment of pulmonary inflammation and/or airway hyperresponsiveness comprising administration of a corticosteroid and an oligonucleotide targeted to IL-4R alpha. Further provided are compositions comprising a corticosteroid and an IL-4R alpha targeted antisense oligonucleotide.

### CROSS REFERENCE TO RELATED APPLICATIONS

This application claims the benefit of priority to U.S. Provisional Application Ser. No. 60/723,426, filed Oct. 3, 2005. This application is related to international application PCT/US2006/006645, filed Feb. 24, 2006, published as WO 2006/091841, which claims the benefit of priority of U.S. Provisional Patent Application Ser. Nos. 60/656,760, filed on Feb. 25, 2005; 60/688,897, filed Jun. 9, 2005; 60/700,656 filed Jul. 19, 2005; and 60/709,404, filed Aug. 18, 2005. Each application is incorporated herein by reference in its entirety.

### BACKGROUND

The cytokine IL-4 is produced by T helper type 2 (TH2) cells following antigen receptor engagement, and by mast cells and basophils upon cross-linkage of the high-affinity immunoglobulin E (IgE) receptor. IL-4 elicits responses important for protective immunity, allergy, asthma and inhibition of certain types of autoimmunity. The pleiotropic effects of this cytokine depend upon its binding to a receptor complex consisting of the IL-4R alpha chain (also known as IL-4Ra, CD124, and interleukin 4 receptor alpha), which mediates high-affinity binding, and a second signal-transducing transmembrane protein (Kelly-Welch et al., Science, 2003, 300, 1527-1528; Nelms et al., Annu. Rev. Immunol., 1999, 17, 701-738). In hematopoietic cells, IL-4R alpha dimerizes with the common gamma chain first identified as a component of the IL-2 receptor, forming a type I IL-4R complex. Type II IL-4R complexes are formed instead through dimerization with IL-13R alpha1, are present primarily in non-hematopoietic cells, and can also be associated with binding of the cytokine IL-13 (Kelly-Welch et al., Science, 2003, 300, 1527-1528; Nelms et al., Annu. Rev. Immunol., 1999, 17, 701-738; Zurawski et al., Embo J., 1993, 12, 2663-2670). Because the IL-4R alpha chain is required in both cases for IL-4 mediated effects, it is often simply equated with the IL-4 receptor.

Human IL-4R alpha chain was cloned independently by two groups (Galizzi et al., Int. Immunol., 1990, 2, 669-675; Idzerda et al., J. Exp. Med., 1990, 171, 861-873). In one study, the protein showed 53% sequence identity to murine IL-4R alpha and was predicted to contain a 25 amino acid signal peptide, a 207 amino acid external domain, a 24 amino acid transmembrane region, a 569 amino acid cytoplasmic domain, six potential N-linked glycosylation sites (3 of which were conserved in murine sequences) and five conserved cysteines in the extracellular domain (Idzerda et al., J. Exp. Med., 1990, 171, 861-873; Mosley et al., Cell, 1989, 59, 335-348). Another cloning approach revealed 50% and 67% identity between human and mouse IL-4R extracellular domains at the protein and nucleic acid sequence level, respectively, and led to classification of the human IL-4R as a member of the cytokine receptor family, characterized by the presence of four cysteine residues at fixed distances near the N-terminus and a unique sequence motif (WSXWS) located close to the transmembrane domain (Galizzi et al., Int. Immunol, 1990, 2, 669-675).

Cytoplasmic regions of IL-4R subunits associate with tyrosine kinases of the Janus kinase (JAK) family Including JAK1, JAK3 and TYK2. Formation of IL-4R dimers stimulates JAK activity, resulting in phosphorylation of tyrosine residues in the cytoplasmic domain of IL-4R alpha, which function as docking sites for signaling molecules containing phospho-protein tyrosine binding Src homology 2 (SH2) domains and subsequent formation of activated STAT6 homodimers that are able to migrate to the nucleus and bind consensus sequences in promoters of IL-4 and IL-13 regulated genes. STAT6 activity is important for many IL-4 and IL-13 regulated allergic responses, including TH2 differentiation, IgE production, as well as chemokine and mucus production at sites of allergic inflammation, and may also regulate lymphocyte growth and survival (Kelly-Welch et al., Science, 2003, 300, 1527-1528). IL-4R signaling also recruits insulin receptor substrate (IRS) family proteins, leading to signaling events such as activation of PI3 kinase, which is thought to be important for growth, survival, and gene expression regulation in response to IL-4 (Kelly-Welch et al., Science, 2003, 300, 1527-1528).

IL-4R alpha-deficient BALB/c mice exhibit no overt phenotypic abnormalities and have normal lymphocyte numbers and development. Immune responses in these mice have been analyzed in several model systems (Gessner and Rollinghoff, Immunobiology, 2000, 201, 285-307). One study showed that signaling through IL-4R alpha is critically important in TH2 cell stimulation of airway mucus production, which contributes to clinical symptoms of asthma, airway obstruction, and mortality (Cohn et al., J. Immunol., 1999, 162, 6178-6183).

Atopy in allergic disease is characterized by the formation of IgE antibody and hypersensitivity upon allergen exposure, underlying disease development in susceptible individuals. Although environmental factors play a role, atopy has a strong genetic predisposition (Hershey et al., N. Engl. J. Med., 1997, 337, 1720-1725). The role of IL-4R alpha in IgE production prompted studies investigating possible gene mutations that may precipitate atopy. The human IL-4R alpha gene was previously localized to 16p11.2-16p12.1 (Pritchard et al., Genomics, 1991, 10, 801-806). Hershey at al. described a polymorphism of this gene that occurred with increased frequency in patients with allergic inflammatory disorders. The variant allele (Q576R) caused a change from glutamine to arginine in the cytoplasmic domain of the receptor (Hershey et al., N. Engl. J. Med., 1997, 337, 1720-1725). Further studies confirmed potential existence of a chromosome 16 susceptibility locus and association of IL-4R alpha gene polymorphisms with atopy (Ober et al., Clin. Exp. Allergy, 1999, 29 Suppl 4, 11-15) (Deichmann et al., Clin. Exp. Allergy, 1998, 28, 151-155) (Kruse et al., Immunology, 1999, 96, 365-371), while other reports suggested that IL-4 gene variations and chromosome 16 were not linked or associated with atopic disease predisposition in certain subject groups (Grimbacher et al., N. Engl. J. Med., 1998, 338, 1073-1074) (Patuzzo et al., J. Med. Genet., 2000, 37, 382-384) (Patuzzo et al., J. Med. Genet., 2000, 37, 382-384) (Haagerup et al., Allergy, 2001, 56, 775-779). Similar studies have linked asthma with IL-4R alpha variants or chromosome 16 (Howard et al., Am. J. Hum. Genet., 2002, 70, 230-236) (Faffe et al., Am. J. Physiol. Lung Cell. Mol. Physiol., 2003, 285, L907-914) (Mitsuyasu et al., Nat. Genet., 1998, 19, 119-120), while another found no single gene effect of IL-4R alpha variants or any other gene on chromosome 16 in children with asthma (Wjst et al., Eur. J. Immunogenet., 2002, 29, 263-268).

Recombinant soluble IL-4 receptor (sIL-4R) has been used in cell culture, animal models, and T cells from allergic patients in attempts to neutralize secreted IL-4 molecules. This approach has also been implemented in humans in phase I/II studies, which reported lung function stabilization in moderate asthma patients (Borish et al., Am. J. Respir. Crit. Care. Med., 1999, 160, 1816-1823).

Dreyfus, et al. discloses the use of an external guide sequence targeting human IL-4R alpha mRNA (Dreyfus et al., Int. Immunopharmacol., 2004, 4, 1015-1027).

U.S. Pre-Grant Publication No. 2004-0049022 discloses compositions and methods for manufacture of single or multiple target antisense oligonucleotides (STA or MTA oligos) of low or no adenosine content for respiratory disease-relevant genes, a method for screening candidate compounds useful for the prevention and/or treatment of respiratory diseases which bind to gene(s), EST(s), cDNA(s), mRNA(s), or their expressed product(s), as well as a list of example nucleic acid targets including interleukin-4 receptor (Nyce et al., 2004).

U.S. Pre-Grant Publication No. 2004-0040052 discloses a method of producing a transgenic cell by introduction of a non-primate lentiviral expression vector with a nucleotide of interest (NOI) capable of generating an antisense oligonucleotide, a ribozyme, an siRNA, a short hairpin RNA, a micro-RNA or a group 1 intron. Disclosed is a list of genes that are associated with human disease, including IL-4R alpha (Radcliffe et al., 2004).

U.S. Pre-Grant Publication No. 2003-0078220 discloses compositions and methods for detecting one or more single nucleotide polymorphisms in the human IL-4R alpha gene and various genotypes and haplotypes for the gene. Design of antisense oligonucleotides to block translation of IL-4R alpha mRNA transcribed from a particular isogene is described (Chew et al., 2003).

The role of IL-4R alpha in inflammatory pathways suggests inhibition of this target gene may be desirable for the treatment of inflammatory diseases, including inflammatory respiratory diseases. Currently, inhaled corticosteroids are often used to treat inflammatory respiratory diseases such as asthma. One such corticosteroid is budesonide (K. R. Chapman, 2003, Clinical Therapeutics 25: C2-C14). However, steroids often have undesirable side effects, creating a need to reduce the amount of steroid used for treatment.

Antisense technology is an effective means for reducing the expression of one or more specific gene products and is uniquely useful in a number of therapeutic, diagnostic, and research applications. Thus, disclosed herein are antisense compounds useful for modulating IL-4R alpha expression and associated pathways via antisense mechanisms of action such as RNaseH, RNAi and dsRNA enzymes, as well as other antisense mechanisms based on target degradation or target occupancy. Methods of treating inflammatory respiratory disease using antisense compounds targeting IL-4R alpha, alone or in combination with a corticosteroid, are described.

Provided herein is a method for prevention, amelioration or treatment of inflammatory respiratory disease, comprising selecting a patient diagnosed with inflammatory respiratory disease and administering to the patient a corticosteroid and an antisense oligonucleotide targeted to IL-4R alpha. Further provided is a method for prevention, amelioration or treatment of inflammatory respiratory disease in a patient in need of such therapy, comprising selecting a patient being treated with a corticosteroid and administering to the patient an antisense oligonucleotide targeted to IL-4R alpha. Also provided is a method for reducing the minimum effective dose of a corticosteroid in a patient diagnosed with inflammatory respiratory disease, comprising selecting a patient being treated with a corticosteroid and administering to the patient the corticosteroid and an antisense oligonucleotide targeted to IL-4R alpha. Further provided are methods for improving one or more symptoms associated with inflammatory respiratory disease in a patient, and for improving inflammatory respiratory disease control in a patient, comprising selecting a patient whose disease is not adequately controlled by corticosteroid treatment and administering to the patient a corticosteroid and an antisense oligonucleotide targeted to IL-4R alpha.

In one embodiment, the inflammatory respiratory disease is asthma, allergic rhinitis, chronic obstructive pulmonary disease or bronchitis.

In another embodiment, the improvement in disease control is measured by a decrease in the number of symptoms, a decrease in the severity of symptoms, a decrease in the duration of symptoms, a decrease in the number of days with symptoms, an inhibition in recurrence of symptoms or a decrease in the dose or frequency of corticosteroid required.

In another embodiment, the symptoms of inflammatory respiratory disease are selected from airway hyperresponsiveness, pulmonary inflammation, mucus accumulation, eosinophil infiltration, increased production of inflammatory cytokines, coughing, sneezing, wheezing, shortness of breath, chest tightness, chest pain, fatigue, runny nose, post-nasal drip, nasal congestion, sore throat, tearing eyes and headache.

In one embodiment, the administering comprises delivery of the corticosteroid and antisense oligonucleotide in a single formulation. In one aspect, the single formulation is delivered by inhalation.

In another embodiment, the administering comprises delivery of the corticosteroid and the antisense oligonucleotide in separate formulations. In one aspect, the separate formulations are delivered simultaneously. In another aspect, the separate formulations are delivered at distinct timepoints. In one aspect, delivery of one or both formulations is by inhalation.

In one embodiment of the methods, the antisense oligonucleotides are 13 to 30 nucleobases in length. In another embodiment, the antisense oligonucleotides are targeted to a region of human IL-4R alpha. In one aspect, the region is at least an 8-nucleobase portion of nucleotides 2056-2087 of human IL-4R alpha (SEQ ID NO: 3). In another aspect, the region is at least an 8-nucleobase portion of nucleotides 2060-2079 of human IL-4R alpha (SEQ ID NO: 3). In one embodiment, the antisense oligonucleotide comprises SEQ ID NO: 25. In another embodiment, the antisense oligonucleotide consists of SEQ ID NO: 25.

In one embodiment of the methods, the corticosteroid is budesonide.

Also provided herein are pharmaceutical compositions comprising a corticosteroid and an antisense oligonucleotide targeted to human IL-4R alpha. In one embodiment, the antisense oligonucleotides are 13 to 30 nucleobases in length. In another embodiment, the antisense oligonucleotides are targeted to a region of human IL-4R alpha. In one aspect, the region is at least an 8-nucleobase portion of nucleotides 2056-2087 of human IL-4R alpha (SEQ ID NO: 3). In another aspect, the region is at least an 8-nucleobase portion of nucleotides 2060-2079 of human IL-4R alpha (SEQ ID NO: 3). In one embodiment, the antisense oligonucleotide comprises SEQ ID NO: 25. In another embodiment, the antisense oligonucleotide consists of SEQ ID NO: 25. In one embodiment, the corticosteroid is budesonide.

Further provided is the use of a pharmaceutical composition comprising a corticosteroid and an antisense oligonucleotide targeted to IL-4R alpha for the preparation of a medicament for prevention, amelioration and/or treatment of airway hyperresponsiveness or pulmonary inflammation. Also provided is the use of an antisense oligonucleotide targeted to IL-4R alpha for the preparation of a medicament for the treatment of inflammatory respiratory disease in a patient being treating with a corticosteroid. Also provided is the use of an antisense oligonucleotide targeted to IL-4R alpha for the preparation of a medicament for the treatment of inflammatory respiratory disease in a patient whose disease is not adequately controlled by corticosteroid treatment. Also provided herein is the use of an antisense oligonucleotide targeted to IL-4R alpha for the preparation of a medicament for reducing the minimum effective dose of a corticosteroid in a patient diagnosed with inflammatory respiratory disease. Further provided is the use of an antisense oligonucleotide targeted to IL-4R alpha for the preparation of a medicament for reducing the dose of corticosteroid required for prevention, amelioration or treatment of inflammatory respiratory disease. In one embodiment, the corticosteroid is budesonide. In another embodiment, the medicament is formulated for delivery by inhalation.

### DETAILED DESCRIPTION

### Overview

There is a large unmet need for satisfactory therapies for a number of inflammatory respiratory diseases including, but not limited to, allergic rhinitis, chronic obstructive pulmonary disease (COPD), asthma and bronchitis. Current therapies, including inhaled corticosteroids, often have undesirable side effects, especially in children. Although many patients with respiratory disease improve with steroid treatment, satisfactory disease management is often not achieved. In addition, it is common for patients being treated with steroids to become sensitized, which leads to an increase in the dose of steroid needed to achieve the same therapeutic effect. Thus, it is desirable to have therapeutic interventions that allow for a decrease in the amount of steroid delivered to patients in need of therapy. It is further desirable to develop treatments to further improve inflammatory respiratory disease control.

Antisense technology is an effective means for reducing the expression of one or more specific gene products and is uniquely useful in a number of therapeutic, diagnostic, and research applications. Provided herein are antisense compounds useful for modulating gene expression and associated pathways via antisense mechanisms of action. The principle behind antisense technology is that an antisense compound, which hybridizes to a target nucleic acid, modulates gene expression activities such as transcription, splicing or translation through one of a number of antisense mechanisms. The sequence specificity of antisense compounds makes them extremely attractive as tools for target validation and gene functionalization, as well as therapeutics to selectively modulate the expression of genes involved in disease.

Disclosed herein are antisense compounds, including antisense oligonucleotides, for use in modulating the expression of nucleic acid molecules encoding IL-4R alpha.

Also provided are methods of preventing, ameliorating or treating inflammatory respiratory disease in a patient by administration of a corticosteroid and an antisense oligonucleotide targeted to IL-4R alpha. In one embodiment, the corticosteroid and antisense oligonucleotide are administered in one formulation. In another embodiment, the corticosteroid and antisense oligonucleotide are prepared in separate formulations and can be administered simultaneously or at distinct timepoints. The corticosteroids can be delivered by any means, including orally or by inhalation. In one embodiment, the antisense oligonucleotide is delivered by inhalation. As described herein, administration of an antisense oligonucleotide targeted to IL-4R alpha in a patient already receiving corticosteroid treatment for inflammatory respiratory disease reduces the minimum effective dose of the corticosteroid, which can lead to a reduction in the dose or frequency of corticosteroid required for treatment. Administration of an IL-4R alpha antisense oligonucleotide to patients diagnosed with inflammatory respiratory disease can be used as an add-on treatment (i.e. can be administered to patients currently receiving corticosteroid treatment) or can be used as a combination treatment with corticosteroid.

Further provided herein are methods of improving one or more symptoms of inflammatory respiratory disease and for improving disease control. In one embodiment, patients who have been receiving corticosteroid treatment, but whose disease is not adequately controlled, are selected for treatment. Selected patients are administered the corticosteroid and an antisense oligonucleotide targeted to IL-4R alpha. In some instances, the patients continue their normal regimen of corticosteroid treatment and IL-4R alpha antisense oligonucleotide treatment is used as an add-on treatment. In another cases, a new regimen is established whereby corticosteroid and antisense oligonucleotide are either co-administered in a single formulation or administered in separate formulations, either at the same time or at different timepoints.

In another embodiment, patients receiving either no prior treatment, or a non-corticosteroid treatment, are selected. As described above, selected patients are administered the corticosteroid and an antisense oligonucleotide targeted to IL-4R alpha, either in a single formulation or in separate formulations.

The antisense oligonucleotides are typically administered by inhalation. When delivered in separate formulations, the corticosteroid can be delivered by any means, including orally or by inhalation.

As used herein, inflammatory respiratory disease includes, but is not limited to, asthma, chronic obstructive pulmonary disease (COPD), allergic rhinitis and bronchitis.

As used herein, an “improvement in disease control” can be measured in a variety of ways, including, but not limited to, a decrease in the number of symptoms, a decrease in the severity of symptoms, a decrease in the duration of symptoms, a decrease in the number of days with symptoms, an inhibition in recurrence of symptoms or a decrease in the dose or frequency of corticosteroid required. Similarly, an improvement in symptoms refers to a decrease in the number of symptoms, a decrease in the severity of symptoms, a decrease in the duration of symptoms, a decrease in the number of days with symptoms and/or an inhibition in recurrence of symptoms.

As used herein, symptoms of inflammatory respiratory disease include, but are not limited to, airway hyperresponsiveness, pulmonary inflammation, mucus accumulation, cosinophil infiltration, increased production of inflammatory cytokines, coughing, sneezing, wheezing, shortness of breath, chest tightness, chest pain, fatigue, runny nose, post-nasal drip, nasal congestion, sore throat, tearing eyes and headache.

As used herein, such terms as “reducing steroid delivery required” and “reducing the amount of steroid needed” refer to a reduction in the dose or frequency of administration of a steroid.

As used herein, “minimum effective dose of a corticosteroid” refers to the lowest dose of the corticosteroid required to achieve a desired effect or therapeutic outcome in a patient, including, but not limited to, a reduction in severity, duration or frequency of one or more symptoms of inflammatory respiratory disease (i.e. an improving one or more symptoms), or prevention or amelioration of inflammatory respiratory disease. The minimum effective dose can also refer to the dose at which an improvement in disease control is observed. As described herein, by administering a corticosteroid, such as budesonide, with an antisense oligonucleotide targeting IL-4R alpha, therapeutic efficacy (e.g., an improvement in symptoms or disease control) can be achieved with lower doses, or less frequent dosing, of the corticosteroid, thus leading to fewer undesirable side effects caused by the corticosteroid.

As used herein, a patient whose inflammatory respiratory disease is not adequately controlled refers to a patient receiving treatment, such as corticosteroid treatment, who has either not responded to treatment or has not responded effectively enough to improve one or more symptoms of disease or to improve disease control.

### Antisense Mechanisms

As used herein, “antisense mechanisms” are all those involving hybridization of a compound with target nucleic acid, wherein the outcome or effect of the hybridization is either target degradation or target occupancy with concomitant stalling of the cellular machinery involving, for example, transcription or splicing.

Target degradation can include an RNase H. RNase H is a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of DNA-like oligonucleotide-mediated inhibition of gene expression.

Target degradation can include RNA interference (RNAi). RNAi is a form of posttranscriptional gene silencing that was initially defined in the nematode, Caenorhabditis elegans, resulting from exposure to double-stranded RNA (dsRNA). In many species the introduction of double-stranded structures, such as double-stranded RNA (dsRNA) molecules, has been shown to induce potent and specific antisense-mediated reduction of the function of a gene or its associated gene products. The RNAi compounds are often referred to as short interfering RNAs or siRNAs. Recently, it has been shown that it is, in fact, the single-stranded RNA oligomers of antisense polarity of the siRNAs which are the potent inducers of RNAi (Tijsterman et al., Science, 2002, 295, 694-697).

Both RNAi compounds (I.e., single- or double-stranded RNA or RNA-like compounds) and single-stranded RNase H-dependent antisense compounds bind to their RNA target by base pairing (i.e., hybridization) and induce site-specific cleavage of the target RNA by specific RNAses; i.e., both are antisense mechanisms (Vickers et al., 2003, J. Biol. Chem., 278, 7108-7118). Double-stranded ribonucleases (dsRases) such as those in the RNase III and ribonuclease L family of enzymes also play a role in RNA target degradation. Double-stranded ribonucleases and oligomeric compounds that trigger them are further described in U.S. Pat. Nos. 5,898,031 and 6,107,094.

### Target Nucleic Acids

As used herein, “targeting” or “targeted to” refer to the process of designing an oligomeric compound such that the compound hybridizes with a selected nucleic acid molecule. Targeting an oligomeric compound to a particular target nucleic acid molecule can be a multistep process. The process usually begins with the identification of a target nucleic acid whose expression is to be modulated. As used herein, the terms “target nucleic acid” and “nucleic acid encoding IL-4R alpha” encompass DNA encoding IL-4R alpha, RNA (including pre-mRNA and mRNA) transcribed from such DNA, and also cDNA derived from such RNA. As disclosed herein, the target nucleic acid encodes IL-4R alpha.

The targeting process usually also includes determination of at least one target region, segment, or site within the target nucleic acid for the antisense interaction to occur such that the desired effect (e.g., modulation of expression) will result. “Region” is defined as a portion of the target nucleic acid having at least one identifiable structure, function, or characteristic. Target regions may include, for example, a particular exon or intron, or may include only selected nucleobases within an exon or intron which are identified as appropriate target regions. Within regions of target nucleic acids are segments. “Segments” are defined as smaller or sub-portions of regions within a target nucleic acid. “Sites,” as used herein, are defined as unique nucleobase positions within a target nucleic acid. As used herein, the “target site” of an oligomeric compound is the 5′-most nucleotide of the target nucleic acid to which the compound binds.

Provided herein are compositions and methods for modulating the expression of IL-4R alpha (also known as IL4-receptor alpha; Interleukin 4 alpha receptor; CD124; IL-4Ra; interleukin 4 receptor alpha chain). Listed in Table 1 are GENBANK® accession numbers of sequences used to design oligomeric compounds targeted to IL-4R alpha. Table 1 also describes features contained within the gene target nucleic acid sequences. Representative features include 5′UTR, start codon, coding sequence (CDS), stop codon, 3 UTR, exon, intron, exon:exon junction, intron:exon junction and exon:intron junction. “Feature start site” and “feature end site” refer to the first (5′-most) and last (3′-most) nucleotide numbers, respectively, of the described feature with respect to the designated sequence. For example, for a sequence containing a start codon comprising the first three nucleotides, “feature start site” is “1” and “feature end site” is “3”.

Oligomeric compounds provided herein include oligomeric compounds which hybridize with one or more target nucleic acid molecules shown in Table 1, as well as oligomeric compounds which hybridize to other nucleic acid molecules encoding IL-4R alpha. The oligomeric compounds may target any region, segment, or site of nucleic acid molecules which encode IL-4R alpha. Suitable target regions, segments, and sites include, but are not limited to, the 5′UTR, the start codon, the stop codon, the coding region, the 3′UTR, the 5′cap region, introns, exons, intron-exon junctions, exon-intron junctions, exon-exon junctions, or any region or segment of nucleotides, or nucleotide site, within the target RNA.

TABLE 1
Human and Mouse IL-4R alpha Sequences

| | | | Feature | Feature | SEQ |
|---|---|---|---|---|---|
| | | | Start | End | ID |
| Species | Genbank # | Feature | Site | Site | NO |
| Human | BM738518.1 | exon | 107 | 130 | 1 |
| Human | BM738518.1 | intron:exon junction | 130 | 131 | 1 |
| Human | BM738518.1 | exon | 342 | 429 | 1 |
| Human | BM738518.1 | start codon | 360 | 362 | 1 |
| Human | BM738518.1 | exon:exon junction | 429 | 430 | 1 |
| Human | nt 18636000 to 18689000 of NT_010393.14 | exon | 1472 | 1495 | 2 |
| Human | nt 18636000 to 18689000 of NT_010393.14 | intron:exon junction | 1495 | 1496 | 2 |
| Human | nt 18636000 to 18689000 of NT_010393.14 | intron | 1496 | 17540 | 2 |
| Human | nt 18636000 to 18689000 of NT_010393.14 | intron:exon junction | 17540 | 17541 | 2 |
| Human | nt 18636000 to 18689000 of NT_010393.14 | exon | 17541 | 17673 | 2 |
| Human | nt 18636000 to 18689000 of NT_010393.14 | intron:exon junction | 17673 | 17674 | 2 |
| Human | nt 18636000 to 18689000 of NT_010393.14 | intron | 17674 | 27660 | 2 |
| Human | nt 18636000 to 18689000 of NT_010393.14 | intron:exon junction | 27660 | 27661 | 2 |
| Human | nt 18636000 to 18689000 of NT_010393.14 | exon | 27661 | 27748 | 2 |
| Human | nt 18636000 to 18689000 of NT_010393.14 | start codon | 27679 | 27681 | 2 |
| Human | nt 18636000 to 18689000 of NT_010393.14 | intron:exon junction | 27748 | 27749 | 2 |
| Human | nt 18636000 to 18689000 of NT_010393.14 | intron | 27749 | 29595 | 2 |
| Human | nt 18636000 to 18689000 of NT_010393.14 | intron:exon junction | 29595 | 29596 | 2 |
| Human | nt 18636000 to 18689000 of NT_010393.14 | exon | 29596 | 29734 | 2 |
| Human | nt 18636000 to 18689000 of NT_010393.14 | intron:exon junction | 29734 | 29735 | 2 |
| Human | nt 18636000 to 18689000 of NT_010393.14 | intron | 29735 | 32343 | 2 |
| Human | nt 18636000 to 18689000 of NT_010393.14 | intron:exon junction | 32343 | 32344 | 2 |
| Human | nt 18636000 to 18689000 of NT_010393.14 | exon | 32344 | 32495 | 2 |
| Human | nt 18636000 to 18689000 of NT_010393.14 | intron:exon junction | 32495 | 32496 | 2 |
| Human | nt 18636000 to 18689000 of NT_010393.14 | intron | 32496 | 33941 | 2 |
| Human | nt 18636000 to 18689000 of NT_010393.14 | intron:exon junction | 33941 | 33942 | 2 |
| Human | nt 18636000 to 18689000 of NT_010393.14 | exon | 33942 | 34093 | 2 |
| Human | nt 18636000 to 18689000 of NT_010393.14 | intron:exon junction | 34093 | 34094 | 2 |
| Human | nt 18636000 to 18689000 of NT_010393.14 | intron | 34094 | 40014 | 2 |
| Human | nt 18636000 to 18689000 of NT_010393.14 | intron:exon junction | 40014 | 40015 | 2 |
| Human | nt 18636000 to 18689000 of NT_010393.14 | exon | 40015 | 40171 | 2 |
| Human | nt 18636000 to 18689000 of NT_010393.14 | intron:exon junction | 40171 | 40172 | 2 |
| Human | nt 18636000 to 18689000 of NT_010393.14 | intron | 40172 | 43282 | 2 |
| Human | nt 18636000 to 18689000 of NT_010393.14 | intron:exon junction | 43282 | 43283 | 2 |
| Human | nt 18636000 to 18689000 of NT_010393.14 | exon | 43283 | 43382 | 2 |
| Human | nt 18636000 to 18689000 of NT_010393.14 | intron:exon junction | 43382 | 43383 | 2 |
| Human | nt 18636000 to 18689000 of NT_010393.14 | intron | 43383 | 46390 | 2 |
| Human | nt 18636000 to 18689000 of NT_010393.14 | intron:exon junction | 46390 | 46391 | 2 |
| Human | nt 18636000 to 18689000 of NT_010393.14 | exon | 46391 | 46469 | 2 |
| Human | nt 18636000 to 18689000 of NT_010393.14 | intron:exon junction | 46469 | 46470 | 2 |
| Human | nt 18636000 to 18689000 of NT_010393.14 | intron | 46470 | 48240 | 2 |
| Human | nt 18636000 to 18689000 of NT_010393.14 | intron:exon junction | 48240 | 48241 | 2 |
| Human | nt 18636000 to 18689000 of NT_010393.14 | exon | 48241 | 48290 | 2 |
| Human | nt 18636000 to 18689000 of NT_010393.14 | intron:exon junction | 48290 | 48291 | 2 |
| Human | nt 18636000 to 18689000 of NT_010393.14 | intron | 48291 | 49726 | 2 |
| Human | nt 18636000 to 18689000 of NT_010393.14 | intron:exon junction | 49726 | 49727 | 2 |
| Human | nt 18636000 to 18689000 of NT_010393.14 | exon | 49727 | 52249 | 2 |
| Human | nt 18636000 to 18689000 of NT_010393.14 | stop codon | 51303 | 51305 | 2 |
| Human | nt 18636000 to 18689000 of NT_010393.14 | 3′UTR | 51306 | 52249 | 2 |
| Human | X52425.1 | exon | 1 | 24 | 3 |
| Human | X52425.1 | 5′UTR | 1 | 175 | 3 |
| Human | X52425.1 | exon:exon junction | 24 | 25 | 3 |
| Human | X52425.1 | exon | 25 | 157 | 3 |
| Human | X52425.1 | exon:exon junction | 157 | 158 | 3 |
| Human | X52425.1 | exon | 158 | 245 | 3 |
| Human | X52425.1 | start codon | 176 | 178 | 3 |
| Human | X52425.1 | CDS | 176 | 2653 | 3 |
| Human | X52425.1 | exon:exon junction | 245 | 246 | 3 |
| Human | X52425.1 | exon | 246 | 384 | 3 |
| Human | X52425.1 | exon:exon junction | 384 | 385 | 3 |
| Human | X52425.1 | exon | 385 | 536 | 3 |
| Human | X52425.1 | exon:exon junction | 536 | 537 | 3 |
| Human | X52425.1 | exon | 537 | 688 | 3 |
| Human | X52425.1 | exon:exon junction | 688 | 689 | 3 |
| Human | X52425.1 | exon | 689 | 845 | 3 |
| Human | X52425.1 | exon:exon junction | 845 | 846 | 3 |
| Human | X52425.1 | exon | 846 | 945 | 3 |
| Human | X52425.1 | exon:exon junction | 945 | 946 | 3 |
| Human | X52425.1 | exon | 946 | 1024 | 3 |
| Human | X52425.1 | exon:exon junction | 1024 | 1025 | 3 |
| Human | X52425.1 | exon | 1025 | 1074 | 3 |
| Human | X52425.1 | exon:exon junction | 1074 | 1075 | 3 |
| Human | X52425.1 | exon | 1075 | 3597 | 3 |
| Human | X52425.1 | stop codon | 2651 | 2653 | 3 |
| Human | X52425.1 | 3′UTR | 2654 | 3597 | 3 |
| Mouse | AF000304.1 | exon | 1 | 88 | 4 |
| Mouse | AF000304.1 | start codon | 19 | 21 | 4 |
| Mouse | AF000304.1 | CDS | 19 | 2451 | 4 |
| Mouse | AF000304.1 | exon:exon junction | 88 | 89 | 4 |
| Mouse | AF000304.1 | exon | 89 | 230 | 4 |
| Mouse | AF000304.1 | exon:exon junction | 230 | 231 | 4 |
| Mouse | AF000304.1 | exon | 231 | 382 | 4 |
| Mouse | AF000304.1 | exon:exon junction | 382 | 383 | 4 |
| Mouse | AF000304.1 | exon | 383 | 534 | 4 |
| Mouse | AF000304.1 | exon:exon junction | 534 | 535 | 4 |
| Mouse | AF000304.1 | exon | 535 | 691 | 4 |
| Mouse | AF000304.1 | exon:exon junction | 691 | 692 | 4 |
| Mouse | AF000304.1 | exon | 692 | 791 | 4 |
| Mouse | AF000304.1 | exon:exon junction | 791 | 792 | 4 |
| Mouse | AF000304.1 | exon | 792 | 870 | 4 |
| Mouse | AF000304.1 | exon:exon junction | 870 | 871 | 4 |
| Mouse | AF000304.1 | exon | 871 | 920 | 4 |
| Mouse | AF000304.1 | exon:exon junction | 920 | 921 | 4 |
| Mouse | assembled from M64868.1 and M64879.1 | exon | 996 | 1055 | 5 |
| Mouse | assembled from M64868.1 and M64879.1 | intron:exon junction | 1055 | 1056 | 5 |
| Mouse | assembled from M64868.1 and M64879.1 | intron | 1056 | 1080 | 5 |
| Mouse | assembled from M64868.1 and M64879.1 | exon | 1206 | 1381 | 5 |
| Mouse | assembled from M64868.1 and M64879.1 | intron:exon junction | 1381 | 1382 | 5 |
| Mouse | assembled from M64868.1 and M64879.1 | intron | 1382 | 1406 | 5 |
| Mouse | assembled from M64868.1 and M64879.1 | exon | 1532 | 1619 | 5 |
| Mouse | assembled from M64868.1 and M64879.1 | start codon | 1550 | 1552 | 5 |
| Mouse | assembled from M64868.1 and M64879.1 | intron:exon junction | 1619 | 1620 | 5 |
| Mouse | assembled from M64868.1 and M64879.1 | intron | 1620 | 1644 | 5 |
| Mouse | assembled from M64868.1 and M64879.1 | exon | 1770 | 1911 | 5 |
| Mouse | assembled from M64868.1 and M64879.1 | intron:exon junction | 1911 | 1912 | 5 |
| Mouse | assembled from M64868.1 and M64879.1 | intron | 1912 | 1936 | 5 |
| Mouse | assembled from M64868.1 and M64879.1 | exon | 2062 | 2213 | 5 |
| Mouse | assembled from M64868.1 and M64879.1 | intron:exon junction | 2213 | 2214 | 5 |
| Mouse | assembled from M64868.1 and M64879.1 | intron | 2214 | 2238 | 5 |
| Mouse | assembled from M64868.1 and M64879.1 | exon | 2364 | 2515 | 5 |
| Mouse | assembled from M64868.1 and M64879.1 | intron:exon junction | 2515 | 2516 | 5 |
| Mouse | assembled from M64868.1 and M64879.1 | intron | 2516 | 2540 | 5 |
| Mouse | assembled from M64868.1 and M64879.1 | exon | 2666 | 2822 | 5 |
| Mouse | assembled from M64868.1 and M64879.1 | intron:exon junction | 2822 | 2823 | 5 |
| Mouse | assembled from M64868.1 and M64879.1 | intron | 2823 | 2847 | 5 |
| Mouse | assembled from M64868.1 and M64879.1 | exon | 2973 | 3086 | 5 |
| Mouse | assembled from M64868.1 and M64879.1 | stop codon | 2990 | 2992 | 5 |
| Mouse | assembled from M64868.1 and M64879.1 | intron:exon junction | 3086 | 3087 | 5 |
| Mouse | assembled from M64868.1 and M64879.1 | intron | 3087 | 3111 | 5 |
| Mouse | assembled from M64868.1 and M64879.1 | exon | 3237 | 3336 | 5 |
| Mouse | assembled from M64868.1 and M64879.1 | intron:exon junction | 3336 | 3337 | 5 |
| Mouse | assembled from M64868.1 and M64879.1 | intron | 3337 | 3361 | 5 |
| Mouse | assembled from M64868.1 and M64879.1 | exon | 3487 | 3565 | 5 |
| Mouse | assembled from M64868.1 and M64879.1 | intron:exon junction | 3565 | 3566 | 5 |
| Mouse | assembled from M64868.1 and M64879.1 | intron | 3566 | 3590 | 5 |
| Mouse | assembled from M64868.1 and M64879.1 | exon | 3716 | 3765 | 5 |
| Mouse | assembled from M64868.1 and M64879.1 | intron:exon junction | 3765 | 3766 | 5 |
| Mouse | assembled from M64868.1 and M64879.1 | intron | 3766 | 3790 | 5 |
| Mouse | assembled from M64868.1 and M64879.1 | exon | 3916 | 6358 | 5 |
| Mouse | assembled from M64868.1 and M64879.1 | CDS | 4643 | 5446 | 5 |
| Mouse | assembled from M64868.1 and M64879.1 | 3′UTR | 5447 | 6058 | 5 |
| Mouse | BB867141.1 | exon:exon junction | 58 | 59 | 6 |
| Mouse | BB867141.1 | exon | 59 | 146 | 6 |
| Mouse | BB867141.1 | start codon | 77 | 79 | 6 |
| Mouse | BB867141.1 | exon:exon junction | 146 | 147 | 6 |
| Mouse | BB867141.1 | exon | 147 | 288 | 6 |
| Mouse | BB867141.1 | exon:exon junction | 288 | 289 | 6 |
| Mouse | BB867141.1 | exon | 289 | 440 | 6 |
| Mouse | BB867141.1 | exon:exon junction | 440 | 441 | 6 |
| Mouse | BC012309.1 | CDS | 313 | 1116 | 7 |
| Mouse | BC012309.1 | 3′UTR | 1117 | 1728 | 7 |
| Mouse | M27959.1 | 5′UTR | 1 | 236 | 8 |
| Mouse | M27959.1 | exon:exon junction | 42 | 43 | 8 |
| Mouse | M27959.1 | exon | 43 | 218 | 8 |
| Mouse | M27959.1 | exon:exon junction | 218 | 219 | 8 |
| Mouse | M27959.1 | exon | 219 | 306 | 8 |
| Mouse | M27959.1 | start codon | 237 | 239 | 8 |
| Mouse | M27959.1 | CDS | 237 | 2669 | 8 |
| Mouse | M27959.1 | exon:exon junction | 306 | 307 | 8 |
| Mouse | M27959.1 | exon | 307 | 448 | 8 |
| Mouse | M27959.1 | exon:exon junction | 448 | 449 | 8 |
| Mouse | M27959.1 | exon | 449 | 600 | 8 |
| Mouse | M27959.1 | exon:exon junction | 600 | 601 | 8 |
| Mouse | M27959.1 | exon | 601 | 752 | 8 |
| Mouse | M27959.1 | exon:exon junction | 752 | 753 | 8 |
| Mouse | M27959.1 | exon | 753 | 909 | 8 |
| Mouse | M27959.1 | 3′UTR | 816 | 3583 | 8 |
| Mouse | M27959.1 | exon:exon junction | 909 | 910 | 8 |
| Mouse | M27959.1 | exon | 910 | 1009 | 8 |
| Mouse | M27959.1 | exon:exon junction | 1009 | 1010 | 8 |
| Mouse | M27959.1 | exon | 1010 | 1088 | 8 |
| Mouse | M27959.1 | exon:exon junction | 1088 | 1089 | 8 |
| Mouse | M27959.1 | exon | 1089 | 1138 | 8 |
| Mouse | M27959.1 | exon:exon junction | 1138 | 1139 | 8 |
| Mouse | M27959.1 | 3′UTR | 2670 | 3281 | 8 |
| Mouse | M27960.1 (or NM_010557.1) | 5′UTR | 1 | 236 | 9 |
| Mouse | M27960.1 (or NM_010557.1) | exon:exon junction | 42 | 43 | 9 |
| Mouse | M27960.1 (or NM_010557.1) | exon | 43 | 218 | 9 |
| Mouse | M27960.1 (or NM_010557.1) | exon:exon junction | 218 | 219 | 9 |
| Mouse | M27960.1 (or NM_010557.1) | exon | 219 | 306 | 9 |
| Mouse | M27960.1 (or NM_010557.1) | start codon | 237 | 239 | 9 |
| Mouse | M27960.1 (or NM_010557.1) | CDS | 237 | 929 | 9 |
| Mouse | M27960.1 (or NM_010557.1) | exon:exon junction | 306 | 307 | 9 |
| Mouse | M27960.1 (or NM_010557.1) | exon | 307 | 448 | 9 |
| Mouse | M27960.1 (or NM_010557.1) | exon:exon junction | 448 | 449 | 9 |
| Mouse | M27960.1 (or NM_010557.1) | exon | 449 | 600 | 9 |
| Mouse | M27960.1 (or NM_010557.1) | exon:exon junction | 600 | 601 | 9 |
| Mouse | M27960.1 (or NM_010557.1) | exon | 601 | 752 | 9 |
| Mouse | M27960.1 (or NM_010557.1) | exon:exon junction | 752 | 753 | 9 |
| Mouse | M27960.1 (or NM_010557.1) | exon | 753 | 909 | 9 |
| Mouse | M27960.1 (or NM_010557.1) | exon:exon junction | 909 | 910 | 9 |
| Mouse | M27960.1 (or NM_010557.1) | exon | 910 | 1023 | 9 |
| Mouse | M27960.1 (or NM_010557.1) | stop codon | 927 | 929 | 9 |
| Mouse | M27960.1 (or NM_010557.1) | 3′UTR | 930 | 3697 | 9 |
| Mouse | M27960.1 (or NM_010557.1) | exon:exon junction | 1023 | 1024 | 9 |
| Mouse | M27960.1 (or NM_010557.1) | exon | 1024 | 1123 | 9 |
| Mouse | M27960.1 (or NM_010557.1) | exon:exon junction | 1123 | 1124 | 9 |
| Mouse | M27960.1 (or NM_010557.1) | exon | 1124 | 1202 | 9 |
| Mouse | M27960.1 (or NM_010557.1) | exon:exon junction | 1202 | 1203 | 9 |
| Mouse | M27960.1 (or NM_010557.1) | exon | 1203 | 1252 | 9 |
| Mouse | M27960.1 (or NM_010557.1) | exon:exon junction | 1252 | 1253 | 9 |
| Mouse | M27960.1 (or NM_010557.1) | CDS | 1980 | 2783 | 9 |
| Mouse | M27960.1 (or NM_010557.1) | 3′UTR | 2784 | 3395 | 9 |
| Mouse | M29854.1 | exon:exon junction | 26 | 27 | 10 |
| Mouse | M29854.1 | exon | 27 | 202 | 10 |
| Mouse | M29854.1 | exon:exon junction | 202 | 203 | 10 |
| Mouse | M29854.1 | exon | 203 | 290 | 10 |
| Mouse | M29854.1 | start codon | 221 | 223 | 10 |
| Mouse | M29854.1 | CDS | 221 | 2653 | 10 |
| Mouse | M29854.1 | exon:exon junction | 290 | 291 | 10 |
| Mouse | M29854.1 | exon | 291 | 432 | 10 |
| Mouse | M29854.1 | exon:exon junction | 432 | 433 | 10 |
| Mouse | M29854.1 | exon | 433 | 584 | 10 |
| Mouse | M29854.1 | exon:exon junction | 584 | 585 | 10 |
| Mouse | M29854.1 | exon | 585 | 736 | 10 |
| Mouse | M29854.1 | exon:exon junction | 736 | 737 | 10 |
| Mouse | M29854.1 | exon | 737 | 893 | 10 |
| Mouse | M29854.1 | exon:exon junction | 893 | 894 | 10 |
| Mouse | M29854.1 | exon | 894 | 993 | 10 |
| Mouse | M29854.1 | exon:exon junction | 993 | 994 | 10 |
| Mouse | M29854.1 | exon | 994 | 1072 | 10 |
| Mouse | M29854.1 | exon:exon junction | 1072 | 1073 | 10 |
| Mouse | M29854.1 | exon | 1073 | 1122 | 10 |
| Mouse | M29854.1 | exon:exon junction | 1122 | 1123 | 10 |
| Mouse | M29854.1 | exon | 1123 | 3565 | 10 |
| Mouse | M29854.1 | 3′UTR | 2654 | 3265 | 10 |

### Modulation of Target Expression

Modulation of expression of a target nucleic acid can be achieved through alteration of any number of nucleic acid (DNA or RNA) functions. “Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of IL-4R alpha. Thus, in one embodiment modulation of expression can mean increase or decrease in target RNA or protein levels. In another embodiment modulation of expression can mean an increase or decrease of one or more RNA splice products, or a change in the ratio of two or more splice products.

The effect of oligomeric compounds on target nucleic acid expression can be tested in any of a variety of cell types provided that the target nucleic acid is present at measurable levels. The effect can be routinely determined using, for example, PCR or Northern blot analysis. Cell lines are derived from both normal tissues and cell types and from cells associated with various disorders. Cell lines derived from multiple tissues and species can be obtained from American Type Culture Collection (ATCC, Manassas, Va.) and are well known to those skilled in the art. Primary cells, or those cells which are isolated from an animal and not subjected to continuous culture, can be prepared according to methods known in the art or obtained from various commercial suppliers. Additionally, primary cells include those obtained from donor human subjects in a clinical setting (i.e. blood donors, surgical patients). Primary cells prepared by methods known in the art.

### Assaying Modulation of Expression

Modulation of IL-4R alpha expression can be assayed in a variety of ways known in the art. IL-4R alpha mRNA levels can be quantitated by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Levels of a protein encoded by IL-4R alpha can be quantitated in a variety of ways well known in the art, such as immunoprecipitation, Western blot analysis (immunoblotting), ELISA or fluorescence-activated cell sorting (FACS). Antibodies directed to a protein encoded by IL-4R alpha can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2; pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Kits, Research Reagents and Diagnostics

The antisense compounds provided herein can be utilized for diagnostics, and as research reagents and kits. Furthermore, antisense compounds, which are able to inhibit gene expression or modulate gene expression with specificity, are often used by those of ordinary skill to elucidate the function of particular genes or to distinguish between functions of various members of a biological pathway.

For use in kits and diagnostics, the antisense compounds provided herein, either alone or in combination with other compounds or therapeutics, can be used as tools in differential and/or combinatorial analyses to elucidate expression patterns of a portion or the entire complement of genes expressed within cells and tissues. Methods of gene expression analysis are well known to those skilled in the art.

### Therapeutics

Antisense compounds provided herein can be used to modulate the expression of IL-4R alpha in an animal, such as a human. In one non-limiting embodiment, the methods comprise the step of administering to said animal in need of therapy for a disease or condition associated with TL-4R alpha an effective amount of an antisense compound that modulates expression of IL-4R alpha. A disease or condition associated with IL-4R alpha includes, but is not limited to, airway hyperresponsiveness, pulmonary inflammation, asthma, rhinitis and bronchitis. Antisense compounds that effectively modulate expression of IL-4R alpha RNA or protein products of expression are considered active antisense compounds.

For example, modulation of expression of IL-4R alpha can be measured in a bodily fluid, which may or may not contain cells; tissue; or organ of the animal. Methods of obtaining samples for analysis, such as body fluids (e.g., sputum, serum), tissues (e.g., biopsy), or organs, and methods of preparation of the samples to allow for analysis are well known to those skilled in the art. Methods for analysis of RNA and protein levels are discussed above and are well known to those skilled in the art. The effects of treatment can be assessed by measuring biomarkers associated with the target gene expression in the aforementioned fluids, tissues or organs, collected from an animal contacted with one or more compounds, by routine clinical methods known in the art. These biomarkers include but are not limited to: liver transaminases, bilirubin, albumin, blood urea nitrogen, creatine and other markers of kidney and river function; interleukins, tumor necrosis factors, intracellular adhesion molecules, C-reactive protein, chemokines, cytokines, and other markers of inflammation.

The antisense compounds provided herein can be utilized in pharmaceutical compositions by adding an effective amount of a compound to a suitable pharmaceutically acceptable diluent or carrier. Acceptable carriers and diluents are well known to those skilled in the art. Selection of a diluent or carrier is based on a number of factors, including, but not limited to, the solubility of the compound and the route of administration. Such considerations are well understood by those skilled in the art. The compounds provided herein can also be used in the manufacture of a medicament for the treatment of diseases and disorders related to IL-4R alpha.

Methods whereby bodily fluids, organs or tissues are contacted with an effective amount of one or more of the antisense compounds or compositions are also contemplated. Bodily fluids, organs or tissues can be contacted with one or more of the compounds described herein resulting in modulation of IL-4R alpha expression in the cells of bodily fluids, organs or tissues. An effective amount can be determined by monitoring the modulatory effect of the antisense compound or compounds or compositions on target nucleic acids or their products by methods routine to the skilled artisan.

Thus, provided herein is the use of an isolated antisense compound targeted to IL-4R alpha in the manufacture of a medicament for the treatment of a disease or disorder by means of the method described above.

### Antisense Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides and alternate splicers. In one embodiment, the oligomeric compound comprises an antisense strand hybridized to a sense strand. Oligomeric compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

In one embodiment, double-stranded antisense compounds encompass short interfering RNAs (siRNAs). As used herein, the term “siRNA” is defined as a double-stranded compound having a first and second strand and comprises a central complementary portion between said first and second strands and terminal portions that are optionally complementary between said first and second strands or with the target mRNA. The ends of the strands may be modified by the addition of one or more natural or modified nucleobases to form an overhang. In one nonlimiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand may contain modifications or additions to either terminus. For example, in one embodiment, both strands of the siRNA duplex would be complementary over the central nucleobases, each having overhangs at one or both termini. It is possible for one end of a duplex to be blunt and the other to have overhanging nucleobases.

In one embodiment, the number of overhanging nucleobases is from 1 to 6 on the 3′ end of each strand of the duplex. In another embodiment, the number of overhanging nucleobases is from 1 to 6 on the 3′ end of only one strand of the duplex. In a further embodiment, the number of overhanging nucleobases is from 1 to 6 on one or both 5′ ends of the duplexed strands. In another embodiment, the number of overhanging nucleobases is zero.

In one embodiment, double-stranded antisense compounds are canonical siRNAs. As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang.

The oligomeric compounds provided herein comprise compounds from about 8 to about 80 nucleobases (i.e. from about 8 to about 80 linked nucleosides). One will appreciate that this comprehends antisense compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 nucleobases.

In one embodiment, the antisense compounds comprise 10 to 50 nucleobases. One will appreciate that this embodies antisense compounds of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49 or 50 nucleobases.

In some embodiments, the antisense compounds comprise 13 to 30 nucleobases. One will appreciate that this embodies antisense compounds of 13, 14, 15, 16, 17, 18, 19, 20, 21, 22, 23, 24, 25, 26, 27, 28, 29 or 30 nucleobases.

In one embodiment, the antisense compounds comprise 15 to 25 nucleobases. One will appreciate that this embodies antisense compounds of 15, 16, 17, 18, 19, 20, 21, 22, 23, 24 or 25 nucleobases.

In one embodiment, the antisense compounds comprise 19 to 23 nucleobases. One will appreciate that this embodies antisense compounds of 19, 20, 21, 22 or 23 nucleobases.

In one embodiment, the antisense compounds comprise 23 nucleobases.

In one embodiment, the antisense compounds comprise 22 nucleobases.

In one embodiment, the antisense compounds comprise 21 nucleobases.

In one embodiment, the antisense compounds comprise 20 nucleobases.

In one embodiment, the antisense compounds comprise 19 nucleobases.

Antisense compounds 8-80 nucleobases in length, or any length therewithin, comprising a stretch of at least eight (8) consecutive nucleobases selected from within the illustrative antisense compounds are considered to be suitable antisense compounds.

Compounds provided herein include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Other compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that compounds may be represented by oligonucleotide sequences that comprise at least 8 consecutive nucleobases from an internal portion of the sequence of an illustrative compound, and may extend in either or both directions until the oligonucleotide contains about 8 to about 80 nucleobases.

One having skill in the art armed with the antisense compounds illustrated herein will be able, without undue experimentation, to identify further antisense compounds.

### Validated Target Segments

The locations on the target nucleic acid to which active oligomeric compounds hybridize are herein below referred to as “validated target segments.” As used herein the term “validated target segment” is defined as at least an 8-nucleobase portion (i.e. 8 consecutive nucleobases) of a target region to which an active oligomeric compound is targeted. While not wishing to be bound by theory, it is presently believed that these target segments represent portions of the target nucleic acid which are accessible for hybridization.

Target segments can include DNA or RNA sequences that comprise at least the 8 consecutive nucleobases from the 5′-terminus of a validated target segment (the remaining nucleobases being a consecutive stretch of the same DNA or RNA beginning immediately upstream of the 5′-terminus of the target segment and continuing until the DNA or RNA contains about 8 to about 80 nucleobases). Similarly validated target segments are represented by DNA or RNA sequences that comprise at least the 8 consecutive nucleobases from the 3′-terminus of a validated target segment (the remaining nucleobases being a consecutive stretch of the same DNA or RNA beginning immediately downstream of the 3′-terminus of the target segment and continuing until the DNA or RNA contains about 8 to about 80 nucleobases). It is also understood that a validated oligomeric target segment can be represented by DNA or RNA sequences that comprise at least 8 consecutive nucleobases from an internal portion of the sequence of a validated target segment, and can extend in either or both directions until the oligonucleotide contains about 8 to about 80 nucleobases.

The validated target segments identified herein can be employed in a screen for additional compounds that modulate the expression of IL-4R alpha. “Modulators” are those compounds that modulate the expression of IL-4R alpha and which comprise at least an 8-nucleobase portion (i.e. 8 consecutive nucleobases) which is complementary to a validated target segment. The screening method comprises the steps of contacting a validated target segment of a nucleic acid molecule encoding IL-4R alpha with one or more candidate modulators, and selecting for one or more candidate modulators which perturb the expression of a nucleic acid molecule encoding IL-4R alpha. Once it is shown that the candidate modulator or modulators are capable of modulating the expression of a nucleic acid molecule encoding IL-4R alpha, the modulator can then be employed in further investigative studies of the function of IL-4R alpha, or for use as a research, diagnostic, or therapeutic agent. Modulator compounds of IL-4R alpha can also be identified or further investigated using one or more phenotypic assays, each having measurable endpoints predictive of efficacy in the treatment of a particular disease state or condition. Phenotypic assays, kits and reagents for their use are well known to those skilled in the art.

### Hybridization

“Hybridization” means the pairing of complementary strands of oligomeric compounds. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refer to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

### Complementarity

“Complementarity,” as used herein, refers to the capacity for precise pairing between two nucleobases on one or two oligomeric compound strands. For example, if a nucleobase at a certain position of an compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric, compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds provided herein comprise at least 70%, or at least 75%, or at least 80%, or at least 85%, or at least 90%, or at least 95%, or at least 96%, or at least 97%, or at least 98%, or at least 99% sequence complementarity to a target nucleic acid sequence. For example, an oligomeric compound in which 18 of 20 nucleobases of the antisense compound are complementary to a target nucleic acid, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarily with the target nucleic acid would have 77.8% overall complementarity with the target nucleic acid and would thus fall within the scope of the compounds provided herein. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Identity

Antisense compounds, or a portion thereof, may have a defined percent identity to a SEQ ID NO, or a compound having a specific Isis number. As used herein, a sequence is identical to the sequence disclosed herein if it has the same nucleobase pairing ability. For example, a RNA which contains uracil in place of thymidine in the disclosed sequences would be considered identical as they both pair with adenine. This identity may be over the entire length of the oligomeric compound, or in a portion of the antisense compound (e.g., nucleobases 1-20 of a 27-mer may be compared to a 20-mer to determine percent identity of the oligomeric compound to the SEQ ID NO.) It is understood by those skilled in the art that an antisense compound need not have an identical sequence to those described herein to function similarly to the antisense compound described herein. Shortened versions of antisense compound taught herein, or non-identical versions of the antisense compound taught herein are also contemplated. Non-identical versions are those wherein each base does not have the same pairing activity as the antisense compounds disclosed herein. Bases do not have the same pairing activity by being shorter or having at least one abasic site. Alternatively, a non-identical version can include at least one base replaced with a different base with different pairing activity (e.g., G can be replaced by C, A, or T). Percent identity is calculated according to the number of bases that have identical base pairing corresponding to the SEQ ID NO or antisense compound to which it is being compared. The non-identical bases may be adjacent to each other, dispersed through out the oligonucleotide, or both.

For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 8-mer is 78% identical to 6 the 18-mer. Such calculations are well within the ability of those skilled in the art.

The percent identity is based on the percent of nucleobases in the original sequence present in a portion of the modified sequence. Therefore, a 30 nucleobase antisense compound comprising the full sequence of the complement of a 20 nucleobase active target segment would have a portion of 100% identity with the complement of the 20 nucleobase active target segment, while further comprising an additional 10 nucleobase portion. The complement of an active target segment may constitute a single portion. In a preferred embodiment, the oligonucleotides are at least about 80%, at least about 85%, at least about 90%, at least about 95% or 100% identical to at least a portion of one of the illustrated antisense compounds, or of the complement of the active target segments presented herein.

It is well known by those skilled in the art that it is possible to increase or decrease the length of an antisense compound and/or introduce mismatch bases without eliminating activity. For example, in Woolf et al. (Proc. Natl. Acad. Sci. USA 89:7305-7309, 1992, incorporated herein by reference), a series of ASOs 13-25 nucleobases in length were tested for their ability to induce cleavage of a target RNA. ASOs 25 nucleobases in length with 8 or 11 mismatch bases near the ends of the ASOs were able to direct specific cleavage of the target mRNA, albeit to a lesser extent than the ASOs that contained no mismatches. Similarly, target specific cleavage was achieved using a 13 nucleobase ASOs, including those with 1 or 3 mismatches. Maher and Dolnick (Nuc. Acid. Res. 16:3341-3358, 1988, incorporated herein by reference) tested a series of tandem 14 nucleobase ASOs, and a 28 and 42 nucleobase ASOs comprised of the sequence of two or three of the tandem ASOs, respectively, for their ability to arrest translation of human DHFR in a rabbit reticulocyte assay. Each of the three 14 nucleobase ASOs alone were able to inhibit translation, albeit at a more modest level than the 28 or 42 nucleobase ASOs. It is understood that antisense compounds can vary in length and percent complementarity to the target provided that they maintain the desired activity. Methods to determine desired activity are disclosed herein and well known to those skilled in the art.

### Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage. It is often preferable to include chemical modifications in oligonucleotides to alter their activity. Chemical modifications can alter oligonucleotide activity by, for example: increasing affinity of an antisense oligonucleotide for its target RNA, increasing nuclease resistance, and/or altering the pharmacokinetics of the oligonucleotide. The use of chemistries that increase the affinity of an oligonucleotide for its target can allow for the use of shorter oligonucleotide compounds.

The term “nucleobase” or “heterocyclic base moiety” as used herein, refers to the heterocyclic base portion of a nucleoside. In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable to the compounds described herein. The terms modified nucleobase and nucleobase mimetic can overlap but generally a modified nucleobase refers to a nucleobase that is fairly similar in structure to the parent nucleobase, such as for example a 7-deaza purine, a 5-methyl cytosine, or a G-clamp, whereas a nucleobase mimetic would include more complicated structures, such as for example a tricyclic phenoxazine nucleobase mimetic. Methods for preparation of the above noted modified nucleobases are well known to those skilled in the art.

Antisense compounds provided herein may also contain one or more nucleosides having modified sugar moieties. The furanosyl sugar ring of a nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R₁)(R₂) for the ring oxygen at the 4′-position. Modified sugar moieties are well known and can be used to alter, typically increase, the affinity of the antisense compound for its target and/or increase nuclease resistance. A representative list of preferred modified sugars includes but is not limited to bicyclic modified sugars (BNA's), including LNA and ENA (4′-(CH₂)₂—O-2′ bridge); and substituted sugars, especially 2′-substituted sugars having a 2′-F, 2′-OCH₂ or a 2′-O(CH₂)₂—OCH₃ substituent group. Sugars can also be replaced with sugar mimetic groups among others. Methods for the preparations of modified sugars are well known to those skilled in the art.

The compounds described herein may include internucleoside linking groups that link the nucleosides or otherwise modified monomer units together thereby forming an antisense compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

As used herein the term “mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein the term “nucleoside” includes, nucleosides, abasic nucleosides, modified nucleosides, and nucleosides having mimetic bases and/or sugar groups.

As used herein, the term “oligonucleotide” refers to an oligomeric compound which is an oligomer or polymer of ribonucleic acid (RNA) or deoxyribonucleic acid (DNA). This term includes oligonucleotides composed of naturally- and non-naturally-occurring nucleobases, sugars and covalent internucleoside linkages, possibly further including non-nucleic acid conjugates.

The present disclosure provides compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds provided herein are well known to those skilled in the art.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general a chimeric oligomeric compound will have modified nucleosides that can be in isolated positions or grouped together in regions that will define a particular motif. Any combination of modifications and or mimetic groups can comprise a chimeric oligomeric compound.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

As used herein, the term “fully modified motif” refers to an antisense compound comprising a contiguous sequence of nucleosides wherein essentially each nucleoside is a sugar modified nucleoside having uniform modification.

The compounds described herein contain one or more asymmetric centers and thus give rise to enantiomers, diastereomers, and other stereoisomeric configurations that may be defined, in terms of absolute stereochemistry, as (R) or (S), α or β, or as (D) or (L) such as for amino acids et al. The present disclosure is meant to include all such possible isomers, as well as their racemic and optically pure forms.

In one aspect, antisense compounds are modified by covalent attachment of one or more conjugate groups. Conjugate groups may be attached by reversible or irreversible attachments. Conjugate groups may be attached directly to antisense compounds or by use of a linker. Linkers may be mono- or bifunctional linkers. Such attachment methods and linkers are well known to those skilled in the art. In general, conjugate groups are attached to antisense compounds to modify one or more properties. Such considerations are well known to those skilled in the art.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The disclosure is not limited by the method of antisense compound synthesis.

### Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates. The methods described herein are not limited by the method of oligomer purification.

### Salts, Prodrugs and Bioequivalents

The antisense compounds described herein comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the antisense compounds, pharmaceutically acceptable salts of such prodrugs, and other bioequivalents.

The term “prodrug” indicates a therapeutic agent that is prepared in an inactive or less active form that is converted to an active form (i.e., drag) within the body or cells thereof by the action of endogenous enzymes, chemicals, and/or conditions. In particular, prodrug versions of the oligonucleotides are prepared as SATE ((S-acetyl-2-thioethyl) phosphate) derivatives according to the methods disclosed in WO 93/24510 or WO 94/26764. Prodrugs can also include antisense compounds wherein one or both ends comprise nucleobases that are cleaved (e.g., by incorporating phosphodiester backbone linkages at the ends) to produce the active compound.

The term “pharmaceutically acceptable salts” refers to physiologically and pharmaceutically acceptable salts of the compounds: i.e., salts that retain the desired biological activity of the parent compound and do not impart undesired toxicological effects thereto. Sodium salts of antisense oligonucleotides are useful and are well accepted for therapeutic administration to humans. In another embodiment, sodium salts of dsRNA compounds are also provided.

### Formulations

The antisense compounds described herein may also be admixed, encapsulated, conjugated or otherwise associated with other molecules, molecule structures or mixtures of compounds.

The present disclosure also includes pharmaceutical compositions and formulations which include the antisense compounds described herein. The pharmaceutical compositions may be administered in a number of ways depending upon whether local or systemic treatment is desired and upon the area to be treated. In one embodiment, administration is topical to the surface of the respiratory tract, particularly pulmonary, e.g., by nebulization, inhalation, or insufflation of powders or aerosols, by mouth and/or nose (intratracheal, intranasal, epidermal and transdermal). Other routes of administration including oral or parenteral are possible. Parenteral administration includes intravenous, intraarterial, subcutaneous, intraperitoneal or intramuscular injection or infusion; or intracranial, e.g., intrathecal or intraventricular, administration. Sites of administration are known to those skilled in the art. In one embodiment, the formulation comprises budesonide, an anti-inflammatory synthetic corticosteroid, often used for the treatment of asthma. In one aspect, the formulation comprising budesonide is delivered by inhalation.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery). In a preferred embodiment, the pharmaceutical formulations are prepared for pulmonary administration in an appropriate solvent, e.g., water or normal saline, possibly in a sterile formulation, with carriers or other agents to allow for the formation of droplets of the desired diameter for delivery using inhalers, nasal delivery devices, nebulizers, and other devices for pulmonary delivery. Alternatively, the pharmaceutical formulations may be formulated as dry powders for use in dry powder inhalers.

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

### Combinations

Compositions provided herein can contain two or more antisense compounds. In another related embodiment, compositions can contain one or more antisense compounds, particularly oligonucleotides, targeted to a first nucleic acid and one or more additional antisense compounds targeted to a second nucleic acid target. Alternatively, compositions provided herein can contain two or more antisense compounds targeted to different regions of the same nucleic acid target. Two or more combined compounds may be used together or sequentially. Compositions can also be combined with other non-antisense compound therapeutic agents (e.g., a corticosteroid, such as budesonide).

### Nonlimiting Disclosure and Incorporation by Reference

While certain compounds, compositions and methods provided herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

### Example 1

The effect of oligomeric compounds on target nucleic acid expression was tested in the following cell types.

### A549:

The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

### b.END:

The mouse brain endothelial cell line b.END was obtained from Dr. Werner Risau at the Max Plank Institute (Bad Nauheim, Germany). b.END cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for use in oligomeric compound transfection experiments.

When cells reach appropriate confluency, they are treated with oligonucleotide using Lipofectin™ as described. When cells reached 65-75% confluency, they were treated with oligonucleotide. Oligonucleotide was mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/Ml per 100 Nm oligonucleotide. This transfection mixture was incubated at room temperature for approximately 0.5 hours. For cells grown in 96-well plates, wells were washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

A number of other commercially available transfection reagents are available that can be used with the methods disclosed in the application. These reagents include, but are not limited to Cytofectin™ (Gene Therapy Systems, San Diego, Calif.), Lipofectamine™ (Invitrogen Life Technologies, Carlsbad, Calif.), Oligofectamine (Invitrogen Life Technologies, Carlsbad, Calif.), and FuGENE™ (Roche Diagnostics Corp., Indianapolis, Ind.) using methods provided in the manufacture's instructions. Oligonucleotides can also be delivered to cells by electroporation using methods well known to those skilled in the art.

Control oligonucleotides are used to determine the optimal oligomeric compound concentration for a particular cell line. Furthermore, when oligomeric compounds are tested in oligomeric compound screening experiments or phenotypic assays, control oligonucleotides are tested in parallel. The concentration of oligonucleotide used varies from cell line to cell line.

### Example 2

### Real-Time Quantitative PCR Analysis of IL-4R Alpha mRNA Levels

Quantitation of IL-4R alpha mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 mL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

Probes and primers for use in real-time PCR were designed to hybridize to target-specific sequences. The primers and probes and the target nucleic acid sequences to which they hybridize are presented in Table 2. The target-specific PCR probes have FAM covalently linked to the 5′ end and TAMRA or MGB covalently linked to the 3′ end, where FAM is the fluorescent dye and TAMRA or MGB is the quencher dye.

TABLE 2

Gene target-specific primers and
probes for use in real-time PCR

| | | Target | | | SEQ |
|---|---|---|---|---|---|
| Target | | SEQ ID | Sequence | | ID |
| Name | Species | NO | Description | Sequence (5′ to 3′) | NO |
| IL-4R alpha | Human | 3 | Forward Primer | AATGGTCCCACCAATTGCA | 11 |
| IL-4R alpha | Human | 3 | Reverse Primer | CTCCGTTGTTCTCAGGGATACAC | 12 |
| IL-4R alpha | Human | 3 | Probe | TTTTTCTGCTCTCCGAAGCCC | 13 |
| IL-4R alpha | Human | 3 | Forward Primer | CCTGGAGCAACCCGTATCC | 14 |
| IL-4R alpha | Human | 3 | Reverse Primer | TGCCGGGTCGTTTTCACT | 15 |
| IL-4R alpha | Human | 3 | Probe | TTACCTGTATAATCATCTCACC | 16 |
| | | | | TATGCAGTCAACATTTG | |
| IL-4R alpha | Mouse | 9 | Forward Primer | TCCCATTTTGTCCACCGAATA | 17 |
| IL-4R alpha | Mouse | 9 | Reverse Primer | GTTTCTAGGCCCAGCTTCCA | 18 |
| IL-4R alpha | Mouse | 9 | Probe | TGTCACTCAAGGCTCTCAGCGGTCC | 19 |

### Example 3

### Antisense Inhibition of Human IL-4R Alpha by Oligomeric Compounds

A series of oligomeric compounds was designed to target different regions of human IL-4R alpha RNA, using published sequences cited in Table 1. All compounds are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the target-specific primers and probes shown in Table 2. Data are averages from two experiments in which A549 cells were treated with 85 nM of the compounds using Lipofectin™.

The target sites (5′-most nucleotide of the target sequence to which the antisense oligonucleotide binds) of compounds targeting SEQ ID NO: 2 include nucleotides 8231, 20215, 27651, 47104 and 49717. The target sites of compounds targeting SEQ ID NO: 3 include nucleotides 21, 167, 173, 176, 193, 194, 196, 197, 199, 200, 201, 202, 203, 205, 206, 207, 208, 209, 210, 211, 212, 213, 215, 217, 219, 220, 221, 222, 223, 224, 225, 226, 227, 228, 229, 234, 246, 284, 287, 317, 353, 355, 428, 429, 430, 431, 487, 494, 496, 497, 499, 500, 501, 502, 503, 504, 506, 508, 509, 510, 530, 531, 619, 620, 621, 642, 645, 647, 649, 735, 736, 737, 741, 777, 917, 931, 936, 998, 999, 1000, 1001, 1003, 1004, 1005, 1006, 1053, 1077, 1078, 1079, 1080, 1082, 1083, 1085, 1087, 1088, 1090, 1092, 1093, 1094, 1095, 1096, 1098, 1100, 1160, 1175, 1182, 1184, 1221, 1223, 1224, 1227, 1395, 1397, 1398, 1399, 1400, 1401, 1492, 1499, 1506, 1507, 1508, 1509, 1608, 1670, 1671, 1673, 1674, 1676, 1700, 1701, 1703, 1705, 1706, 1708, 1716, 1777, 1779, 1780, 1781, 1782, 1845, 1976, 1997, 2000, 2038, 2043, 2056, 2057, 2058, 2058, 2059, 2060, 2062, 2064, 2065, 2066, 2067, 2067, 2068, 2082, 2087, 2126, 2128, 2130, 2131, 2230, 2301, 2315, 2390, 2403, 2469, 2524, 2526, 2528, 2529, 2530, 2531, 2532, 2541, 2548, 2569, 2578, 2579, 2626, 2643, 2674, 2731, 2743, 2751, 2763, 2772, 2836, 2856, 2861, 2909, 2915, 2952, 3048, 3053, 3103, 3168, 3198, 3238, 3290, 3297, 3303, 3420, 3432, 3477, 3572 and 3578.

Oligonucleotides targeted to the following nucleotide segments of SEQ ID NO: 3 were effective at inhibiting the expression of human IL 4R-o at least about 19%: nucleotides 167-265; 284-306; 317-336; 353-374; 428-450; 487-525; 530-550; 619-640; 642-668; 735-760; 777-796; 917-955; 998-1025; 1053-1072; 1077-1119; 1160-1203; 1221-1246; 1395-1420; 1492-1528; 1608-1627; 1670-1695; 1700-1735; 1777-1801; 1845-1864; 1976-1995; 1997-2019; 2038-2106; 2056-2087; 2126-2150; 2230-2249; 2301-2334; 2390-2422; 2469-2488; 2524-2567; 2569-2598; 2626-2662; 2674-2693; 2731-2791; 2856-2880; 2909-2934; 2952-2971; 3048-3072; 3103-3122; 3168-3187; 3198-3217; 3297-3322; 3420-3451; and 3477-3496. Oligonucleotides targeted to the following nucleotides of SEQ ID NO: 2 were effective at inhibiting the expression of human IL 4R-α at least about 35%: nucleotides 8231-8250; 20215-20234; 27651-27670; and 47104-47123.

### Example 4

### Antisense Inhibition of Mouse IL-4R Alpha by Oligomeric Compounds

A series of oligomeric compounds was designed to target different regions of mouse IL-4R alpha RNA, using SEQ ID NO: 5. All compounds are chimeric oligonucleotides (“gapmers”) 20 nucleotides in length, composed of a central “gap” region consisting of ten 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the target-specific primers and probes shown in Table 2. Data are averages from two experiments in which b.END cells were treated with 150 nM of the compounds using Lipofectin™.

All oligonucleotides targeted to the following nucleotide segments of SEQ ID NO: 5 were effective at inhibiting expression of IL 4R-α at least 40%: nucleotides 2506-2525 and 2804-2323. All oligonucleotides targeted to the following nucleotide segments of SEQ ID NO: 9 were effective at inhibiting expression of IL 4R-α at least 40%: nucleotides 78-97; 233-263; 330-349; 388-407; 443-462; 611-630; 716-740; 758-777; 918-9937; 1014-1033; 1114-1133; 1136-1155; 1385-1314; 1424-1459; 1505-1534; 1575-1594; 1834-1863; 1880-1899; 1991-2030; 2979-2103; 2166-2185; 2437-2461; 2469-2488; 2497-2526; 2719-2738; 2788-2817; 2827-2846; 2859-2888; 3345-3374; and 3671-3697.

### Example 5

### Design and Screening of Duplexed Oligomeric Compounds Targeting IL-4R Alpha

In accordance with provided disclosure, a series of duplexes, including dsRNA and mimetics thereof, comprising oligomeric compounds and their complements can be designed to target IL-4R alpha. The nucleobase sequence of the antisense strand of the duplex comprises at least a portion of an oligonucleotide targeted to IL-4R alpha as disclosed herein. The ends of the strands may be modified by the addition of one or more natural or modified nucleobases to form an overhang. The sense strand of the nucleic acid duplex is then designed and synthesized as the complement of the antisense strand and may also contain modifications or additions to either terminus. The antisense and sense strands of the duplex comprise from about 17 to 25 nucleotides, or from about 19 to 23 nucleotides. Alternatively, the antisense and sense strands comprise 20, 21 or 22 nucleotides.

For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO: 20) and having a two-nucleobase overhang of deoxythyrnidine(dT) would have the following structure:

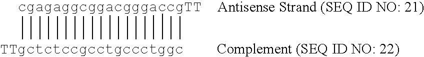

Overhangs can range from 2 to 6 nucleobases and these nucleobases may or may not be complementary to the target nucleic acid. In another embodiment, the duplexes can have an overhang on only one terminus.

In another embodiment, a duplex comprising an antisense strand having the same sequence, for example CGAGAGGCGGACGGGACCG (SEQ ID NO: 20), can be prepared with blunt ends (no single stranded overhang) as shown:

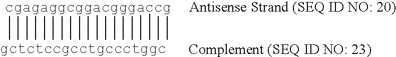

The RNA duplex can be unimolecular or bimolecular; i.e., the two strands can be part of a single molecule or may be separate molecules.

RNA strands of the duplex can be synthesized by methods routine to the skilled artisan or purchased from Dharmacon Research Inc. (Lafayette, Colo.). Once synthesized, the complementary strands are annealed. The single strands are aliquotted and diluted to a concentration of 50. Once diluted, 30 μL of each strand is combined with 15 μL of a 5× solution of annealing buffer. The final concentration of said buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM.

Once prepared, the duplexed compounds are evaluated for their ability to modulate IL-4R alpha. When cells reach 80% confluency, they are treated with the duplexed compounds. For cells grown in 96-well plates, wells are washed once with 200 μL OPTI-MEM-1™ reduced-serum medium (Gibco BRL) and then treated with 130 μL of OPTI-MEM-1™ containing 12 μg/mL LIPOFECTIN™ (Gibco BRL) and the desired duplex antisense compound at a final concentration of 200 nM (a ratio of 6 μg/mL LIPOFECTIN™ per 100 nM duplex antisense compound). After 5 hours of treatment, the medium is replaced with fresh medium. Cells are harvested 16 hours after treatment, at which time RNA is isolated and target reduction measured by RT-PCR.

### Example 6

### Mouse Model of Allergic Inflammation

Based on the in vitro screen described in Example 4, a lead antisense oligonucleotide targeted to mouse IL-4R alpha (ISIS 231894; CCGCTGTTCTCAGGTGACAT; SEQ ID NO: 24) was chosen for testing in in vivo mouse model systems. Compared to a mismatch control oligonucleotide, ISIS 231894 caused dose-dependent mouse IL-4R alpha mRNA reduction 24 hours following treatment of mouse b.END cells (Table 3).

TABLE 3
Dose-dependent reduction of IL-4R alpha mRNA in
mouse b.END cells (% of untreated control)

| Oligonucleotide | | Mismatch |
|---|---|---|
| dose (nM) | ISIS 231894 | Control |
| 0 | 100 | 100 |
| 1 | 100 | 110 |
| 5 | 55 | 120 |
| 10 | 41 | 120 |
| 25 | 35 | 105 |
| 50 | 20 | 95 |
| 100 | 20 | 100 |

In the mouse model of allergic inflammation, mice are sensitized and challenged with aerosolized chicken ovalbumin (OVA). Airway responsiveness is assessed by inducing airflow obstruction using a noninvasive method whereby unrestrained conscious OVA sensitized mice are placed into the main chamber of a plethysmograph (Buxco Electronics, Inc. Troy, N.Y.) and challenged with aerosolized methacholine. Pressure difference between this chamber and a reference chamber is used to extrapolate minute volume, breathing frequency and enhanced pause (Penh). Penh is a dimensionless parameter that is a function of total pulmonary airflow (i.e. the sum of the airflow in the upper and lower respiratory tracts) during the respiratory cycle of a mouse and is lower when airflow is greater. This parameter closely correlates with lung resistance as measured by traditional, invasive techniques using ventilated animals (Hamelmann et al., 1997, Am. J. Respir. Crit. Care Med. 156:766-775).

Several important features common to disease in human asthma and the mouse model of allergic inflammation include pulmonary inflammation, goblet cell hyperplasia and airway hyperresponsiveness (AHR). Pulmonary inflammation is dominated by cytokine expression with a TH2 profile, while goblet cell hyperplasia is a measure of increased mucus production in the mouse, and A-R involves increased sensitivity to cholinergic receptor agonists such as acetylcholine or methacholine. The compositions and methods provided herein may be used to treat AHR and pulmonary inflammation in animals, including humans. The combined use of antisense oligonucleotides to human IL-4R alpha with one or more conventional asthma medications is contemplated.

The mouse model of allergic inflammation was used to test the efficacy of an inhaled antisense oligonucleotide targeted to mouse IL-4R alpha. A mismatched IL-4R alpha oligonucleotide (mismatch control oligonucleotide) was used as a negative control. Male Balb/c mice 8-10 weeks old (Charles River Laboratory, Taconic Farms, N.Y.) were maintained in micro-isolator cages housed in a specific pathogen free (SPF) facility. The sentinel cages within the animal colony surveyed negative for viral antibodies and the presence of known mouse pathogens.

### Ovalbumin Induced Allergic Inflammation-Acute Model

For the acute model of allergic inflammation, mice were sensitized with 20 μg of alum-precipitated OVA was injected intraperitoneally on days 0 and 14. On days 24, 25 and 26, animals were exposed for 20 minutes to 1% OVA (in saline) by ultrasonic nebulization. On days 17, 19, 21, 24 and 26 animals were dosed with vehicle alone (saline), 1 μg/kg or 10 μg/kg of ISIS 231894 or the mismatch control oligonucleotide. Oligonucleotides or vehicle were suspended in 0.9% sodium chloride and delivered via inhalation using a nose-only aerosol delivery exposure system. A Lovelace nebulizer set at a flow rate of 1.4 liter per minute feeding into a total flow rate of 10 liters per minute was used to deliver the oligonucleotide. The exposure chamber was equilibrated with an oligonucleotide aerosol solution for 5 minutes before mice were placed in a restraint tube attached to the chamber. Restrained mice were treated for a total of 10 minutes. Analysis was performed on day 28. The results are shown in Table 4.

TABLE 4
AHR and BAL eosinophil infiltration in acute allergic
inflammation mouse model

| | Penh | % |
|---|---|---|
| Treatment | (100 mg/mL methacholine) | Eosinophils |
| Naïve | 4 | 0 |
| Vehicle | 8 | 65 |
| ISIS 231894- 1 μg/kg | 6 | 35 |
| ISIS 231894- 10 μg/kg | 4.5 | 35 |
| Mismatch control - 1 μg/kg | 9 | 65 |
| Mismatch control- 10 μg/kg | 7 | 55 |

ISIS 231894, but not the mismatch control oligonucleotide, caused a significant, dose dependent suppression in methacholine-induced AHR in sensitized mice as measured through whole body plethysmography and the Penh parameter. Significant improvement in pulmonary function by ISIS 231894 but not the mismatch control was also observed when measuring lung resistance and compliance.

Treatment with ISIS 231894, but not the mismatch control, also resulted in a significant decrease in eosinophil infiltration as determined by cell differentials performed on bronchoalveolar lavage (BAL) fluid collected from lungs of the treated mice after injection of a lethal dose of ketamine. Dendritic cells, eosinophils, macrophages and epithelial cells recovered from collagenase digested lung were analyzed for expression of IL-4R alpha protein by flow cytometry. An oligonucleotide-specific significant reduction of IL-4R alpha protein was seen in the dendritic and epithelial cells as well as the mixed eosinophil and macrophage population from mice treated with ISIS 231894. A second experiment, in which mice were dosed with 10 μg/kg ISIS 231894, confirmed the efficacy of ISIS 231894 to decrease AHR and eosinophilia in the acute model.

The minimum lung tissue concentration of ISIS 231894 was determined to be less than 10 ng/gram (1 to 10 μg/kg estimated inhaled dose). Other in vivo studies showed that intrapulmonary aerosol doses up to 1 mg/kg were well-tolerated in mice and the half life in the lung of ISIS 231894 was estimated to be 2-4 days. Furthermore, once weekly dosing sustained the TL-4R alpha antisense effect and reduced AHR and airway inflammation in mice with well established allergen-induced pulmonary inflammation.

These data demonstrate that IL-4R alpha is a valid target for the prevention, amelioration and/or treatment of diseases associated with AHR and lung inflammation, including asthma and chronic obstructive pulmonary disease (COPD).

### Mouse Model of Allergic Inflammation-Rechallenge Model

The rechallenge model of allergic inflammation includes a second series of nebulized OVA challenges on days 66 and 67 in addition to the sensitization and challenge steps of the acute model. This model allows for the evaluation of the target's role in a recall response, as opposed to its role as an initiator molecule. In the rechallenge model, mice were treated with 10, 100 or 500 μg/kg of either ISIS 231894 or the mismatch control oligonucleotide on days 52, 54, 56, 59 and 61, subsequent to the onset and resolution of the OVA-induced acute inflammatory response, delivered by nose only inhalation. The study endpoints were similar to those in the acute model, and included Penh response (i.e. AHR reduction), inflammatory cells and cytokines in BAL (determined by ELISA), mucus accumulation (as determined by periodic acid-Schiff base [PAS] staining in lungs), lung histology and IL-4R alpha protein reduction in lung epithelial and inflammatory cells (as determined by flow cytometry). The results are shown below in Tables 5 and 6.

TABLE 5
AHR and BAL eosinophil infiltration in allergic
inflammation rechallenge mouse model

| | Penh | % |
|---|---|---|
| Treatment | (100 mg/mL methacholine) | Eosinophils |
| Naïve | 3 | 1 |
| Vehicle | 6 | 37 |
| ISIS 231894- 10 μg/kg | 3 | 22 |
| ISIS 231894- 100 μg/kg | 3.5 | 18 |
| ISIS 231894- 500 μg/kg | 3.5 | 15 |
| Mismatch control- 10 μg/kg | 7 | 35 |
| Mismatch control- 100 μg/kg | 6 | 36 |
| Mismatch control- 500 μg/kg | 4.5 | 33 |

A significant reduction in methacholine-induced AHR (Penh) was observed in response to all three doses of ISIS 231894 as well as in the high dose mismatch control group as compared to vehicle control treated animals. In addition, the percentage of eosinophils in BAL fluid was significantly reduced as compared to treatment with mismatch control oligonucleotide.

TABLE 6
Dose-dependent reduction of target protein in
Rechallenge model (% positive cells)

| | Dendritic | Macrophages/ |
|---|---|---|
| Treatment | cells | Eosinophils |
| Naïve | 18 | 16 |
| Vehicle | 19 | 32 |
| ISIS 231894- 10 μg/kg | 25 | 18 |
| ISIS 231894- 100 μg/kg | 18 | 20 |
| ISIS 231894- 500 μg/kg | 10 | 17 |
| Mismatch control- 10 μg/kg | 22 | 27 |
| Mismatch control - 100 μg/kg | 20 | 31 |
| Mismatch control- 500 μg/kg | 30 | 30 |

Treatment with ISIS 231894, but not the mismatch control also reduced the amount of IL-4R alpha surface expression (determined by flow cytometry) on lung eosinophils macrophages and dendritic cells.

In addition, lung IL-5 mRNA was inhibited at 10 μg and 100 μg doses of ISIS 231894. Treatment with ISIS 231894 also significantly reduced expression of a number of cytokines tested including the inflammatory indicator KC (mouse homologue of IL-8, MCP-1, and the TH2 cytokines IL-5 and IL-13, in the BAL fluid at doses of 100 μg and 500 μg of the oligonucleotide as compared to vehicle control. Together, these data demonstrate that an IL-4R alpha targeted antisense oligonucleotide approach is efficacious in the setting of an immunological recall inflammatory response in the mouse.

### Mouse Model of Allergic Inflammation—Chronic Model

In the chronic model of allergic inflammation, mice are subjected to repeated intranasal OVA administration, producing a chronic inflammatory response. In this model, mice were sensitized by intraperitoneal injection with 100 μg of OVA on days 0 and 14 as in the previous models. OVA was administered at a dose of 500 μg on days 14, 27, 28, 29, 47, 61, 73, 74 and 75. Oligonucleotide, either ISIS 231894 or the mismatch control, was administered via the nose-only aerosol delivery exposure system at a dose of either 5 μg/kg or 500 μg/kg on days 31, 38, 45, 52, 59, 66 and 73. Dexamethasone, an anti-inflammatory agent, was administered by intraperitoneal injection at 2.5 mg/kg on days 47, 62, 73, 74 and 75. Analysis of endpoints was performed on day 76, except cytokines which were evaluated on day 62, 6 hours post OVA challenge. Endpoints were similar to those in the acute and rechallenge model, and included Penh (AHR), BAL inflammatory cell accumulation and cytokines and mucus accumulation. The results are described below and shown in Table 7.

TABLE 7
BAL cell infiltration in chronic allergic inflammation mouse model

| Treatment | % Eosinophils | % Neutrophils |
|---|---|---|
| Naïve | 2 | 6 |
| Vehicle | 49 | 56 |
| Vehicle + dexamethasone | 25 | 58 |
| ISIS 231894- 5 μg/kg | 45 | 38 |
| ISIS 231894- 500 μg/kg | 29 | 35 |

Treatment of mice with each dose of ISIS 231894 or with dexamethasone resulted in a significant decrease in methacholine-induced AHR (Penh) as compared to treatment with vehicle (i.e. saline). In addition, treatment of mice with 500 μg/kg of ISIS 231894 or dexamethasone resulted in a significant decrease in the percent of eosinophils in BAL fluid as compared to vehicle control. Both doses of ISIS 231894 significantly reduced the percent neutrophils in BAL, whereas dexamethasone did not decrease BAL neutrophils. Analysis of BAL fluid also revealed a significant reduction in IL-5 and KC in both 500 μg/kg ISIS 231894 and dexamethasone treated animals as compared to vehicle treated animals. These data demonstrate activity of an inhaled IL-4R alpha antisense oligonucleotide in a mouse model of asthma using a therapeutic administration schedule.

### Example 7

### Inhaled Budesonide and IL-4R Alpha Antisense Oligonucleotide in the Allergic Inflammation Mouse Model

Budesonide is an inhaled corticosteroid used for treatment of respiratory diseases, including allergic rhinitis, asthma and bronchitis. Budesonide acts chiefly by suppressing pulmonary inflammation and reducing airway hyperresponsiveness. The acute mouse model of allergic inflammation was used to determine if co-administration of inhaled IL-4R alpha antisense oligonucleotide would enhance the activity of inhaled budesonide, or reduce the dose required to produce anti-inflammatory activity. As described in Example 6, mice were sensitized with alum-precipitated OVA at day 0 and day 14 and nebulized with OVA in saline on days 24, 25 and 26. All mice were analyzed on day 28. Budesonide (0.3, 3, 30, and 300 μg/kg dissolved in PBS Containing 20% DMSO) was administered by nose-only aerosol exposure beginning on day 23 (24 and 20 hours before OVA exposure) and then daily through day 26, one hour prior to daily OVA exposure. The 30 μg/kg dose was also administered twice a day (bid) from day 23-26 as a separate group. As in Example 6, ISIS 231894 was administered by nose-only aerosol exposure at day 17, 19, 21, 23 and 26. Endpoints were similar to those described in Example 6. The results of treatment with budesonide alone on AHR and BAL cosinophil infiltration are shown below in Table 8.

TABLE 8
AHR and BAL eosinophil infiltration in acute allergic
inflammation model with inhaled budesonide

| | Penh | |
|---|---|---|
| Treatment | (100 mg/mL methacholine) | % Eosinophils |
| Naïve | 3.5 | 0 |
| Vehicle | 5.5 | 45 |
| 30 μg/kg budesonide bid | 3.25 | 20 |
| 300 μg/kg budesonide | 3.75 | 15 |
| 30 μg/kg budesonide | 3.25 | 21 |
| 3 μg/kg budesonide | 4.5 | 32 |
| 0.3 μg/kg budesonide | 4.75 | 41 |

Doses of 30 and 300 μg/kg budesonide induced significant improvement in Penh, BAL eosinophil accumulation and mucus accumulation compared with administration of vehicle alone, suggesting that 30 μg/kg is the minimum effective dose.

To determine whether co-administration of IL-4R alpha antisense oligonucleotide would enhance the activity of budesonide and reduce its minimum effective dose, mice were treated wither either 3 or 30 μg/kg of budesonide with or without 1 μg/kg ISIS 231894. The effect of budesonide and/or ISIS 231894 treatment on AHR, BAL eosinophil infiltration and mucus accumulation (number of PAS-positive airways) were determined. The results are shown in Table 9.

TABLE 9
AHR, BAL eosinophil infiltration and mucus accumulation
(PAS+ airways) in acute allergic inflammation model
with inhaled budesonide and inhaled ISIS 231894

| | Penh | | |
|---|---|---|---|
| | (100 mg/mL | | PAS+ |
| Treatment | methacholine) | % Eosinophils | airways |
| Naïve | 3.3 | 0 | 0 |
| Vehicle | 6 | 41 | 35 |
| 30 μg/kg budesonide | 5.5 | 20 | 18 |
| 1 μg/kg ISIS 231894 | 6.2 | 27 | 21 |
| 30 μg/kg budesonide + | 3.5 | 8 | 12 |
| 1 μg/kg ISIS 231894 | | | |
| 3 μg/kg budesonide + | 4.1 | 23 | 18 |
| 1 μg/kg ISIS 231894 | | | |

When 1 μg/kg ISIS 231894 was co-administered with 3 or 30 μg/kg budesonide, significant changes were observed in Penh compared to saline (vehicle) treatment or either budesonide or IL-4R alpha antisense treatment alone, indicating that co-administration of IL-4R alpha antisense can improve the activity of budesonide in a mouse pulmonary inflammation model. Similar activity of the combination at 3 μg/kg budesonide demonstrates that co-administration of inhaled IL-4R alpha antisense also reduces the efficacious dose of budesonide. Additionally, treatment with 30 μg/kg budesonide in combination with 1 μg/kg ISIS 231894 was significantly more effective at reducing BAL eosinophil percentages and mucus accumulation than either 30 μg/kg budesonide or 1 μg/kg ISIS 231894 alone. These data demonstrate that the two compounds produced additive results for mucus production and BAL eosinophilia and may act synergistically with regard to Penh.

### Example 8

### Intranasal Administration of Budesonide and ISIS 231894 in the Allergic Rhinitis Mouse Model

In a mouse model of allergic rhinitis, animals were sensitized intraperitoneally with alum-precipitated OVA on days 1, 5, 10 and 15. OVA diluted with saline was administered intranasally (25 μL of 500 μg OVA in each nare) daily, on days 18-22, 25-29, and 32-35. ISIS 231894 and budesonide were administered intranasally, with budesonide administration one hour before each intranasal OVA challenge. ISIS 231894 was administered on days 11, 13, 15, and one hour before each intranasal OVA challenge. Endpoints were evaluated on day 36 and included nasal mucus accumulation (nasal histopathology) nasal eosinophilia, neutrophilia (by nasal lavage analysis and microscopic eosinophil counts in epithelial tissue) and allergic symptoms (numbers of sneezes and nose-rubs observed over a fixed time period). The results are shown in Tables 10 and 11.

TABLE 10
Nasal lavage leukocytes and allergic symptoms in allergic
rhinitis model with intranasal budesonide

| | % | % | Nasal rubs | Sneezes |
|---|---|---|---|---|
| | Nasal lavage | Nasal lavage | (per | (per |
| Treatment | neutrophils | eosinophils | 5 min) | 5 min) |
| Naïve | 2 | 1 | 0 | 2 |
| Vehicle | 18 | 16 | 6 | 13 |
| 500 μg/kg budesonide | 6 | 2 | 2 | 2 |
| 350 μg/kg budesonide | 12 | 4 | 4 | 3 |
| 35 μg/kg budesonide | 28 | 11 | 4.5 | 2 |
| 3.5 μg/kg budesonide | ND | ND | 5 | 6 |

TABLE 11
Allergic rhinitis model with intranasal budesonide and ISIS 231894

| | % Nasal | % Nasal | Tissue | | |
|---|---|---|---|---|---|
| | lavage | lavage | eosinophils | Nasal rubs | Sneezes |
| Treatment | neutrophils | eosinophils | (per mm²) | (per 5 min) | (per 5 min) |
| Naïve | 17 | 2 | 0 | 0.2 | 1.6 |
| Vehicle | 16 | 26 | 342 | 4.6 | 8.9 |
| Vehicle + 5% DMSO | 8 | 15 | 371 | 2.4 | 5.7 |
| 35 μg/kg Budesonide + 20% DMSO | 12 | 7 | | 1.2 | 5.8 |
| 35 μg/kg Budesonide + 5% DMSO | 9 | 7 | 174 | 0.6 | 2.8 |
| 0.01 mg/kg Isis 231894 | 16 | 12 | 438 | 0.8 | 2.5 |
| 0.1 mg/kg Isis 231894 | 5 | 2 | 240 | 0.3 | 1.9 |
| 1 mg/kg Isis 231894 | 13 | 5 | 101 | 0.5 | 1.0 |
| 10 mg/kg Isis 231894 | 14 | 9 | 298 | 1.1 | 4.8 |
| 0.1 mg/kg Isis 231894 + 35 μg/kg | 5 | 4 | 169 | 0.4 | 2.9 |
| Budesonide + 5% DMSO | | | | | |
| 1 mg/kg Isis 231894 + 35 μg/kg | 7 | 3 | 102 | 0.9 | 4.3 |
| Budesonide + 5% DMSO | | | | | |

The results demonstrate that intranasal administration of ISIS 231894 or budesonide alone and in combination therapy reduce nasal eosinophilia, neutrophilia and allergic symptoms (sneezes and nose rubs) in this model.

### Example 9

### Human IL-4R Alpha Antisense Oligonucleotides

To further evaluate compounds that actively inhibited human IL-4R alpha (see Example 3), additional studies were conducted in human A549 epithelial cell lines as well as primary small airway epithelial cells focusing specifically on 4 antisense oligonucleotides (ASOs): ASO1, ASO2, ASO3 (TGGAAAGGCTTATACCCCTC; SEQ ID NO: 25) and ASO4. The result of oligonucleotide treatment of A549 cells on IL-4R alpha mRNA is shown in Table 12.

TABLE 12
Dose-dependent reduction of IL-4R alpha mRNA in human
A549 cells (% of untreated control)

| Oligonucleotide dose | Mismatch | | | | |
|---|---|---|---|---|---|
| (nM) | Control | ASO1 | ASO2 | ASO3 | ASO4 |
| 100 | 124 | 12 | 8 | 17 | 13 |
| 50 | 140 | 17 | 11 | 29 | 32 |
| 25 | 109 | 23 | 28 | 45 | 63 |

In both cell types, at concentrations of 100 nM, 50 nM and 25 nM, ASOs 1-4 each caused dose-dependent reduction of target (TL-4R alpha) mRNA and protein (as measured by flow cytometry) with no significant effect on total cellular mRNA, measured 24 hours following ASO treatment. Further, in primary cells, all four compounds caused reduction of cytokine-induced MUC2 mRNA (Table 13), demonstrating that they induced inhibition of human IL-4R alpha activity.

TABLE 13
Dose-dependent reduction of MUC-2 mRNA in human
A549 cells (% of untreated control)

| Oligonucleotide dose | Mismatch | | | | |
|---|---|---|---|---|---|
| (nM) | Control | ASO1 | ASO2 | ASO3 | ASO4 |
| 100 | 170 | 42 | 52 | 39 | 15 |
| 50 | 141 | 58 | 68 | 63 | 56 |
| 25 | 119 | 92 | 90 | 86 | 95 |

Based on these findings, ASO3 was chosen to test for in vivo tolerability in mice. Compared with control animals, mice receiving ASO3 via either nose-only aerosol administration (1, 10, and 100 mg/kg, 3×/week) or systemic (intraperitoneal) injection (10, 60, 100 mg/kg, 2×/week) over a period of three weeks exhibited neither increase in baseline Penh nor an increase in neutrophils or lymphocytes in the lung. Treated animals also demonstrated no change in serum chemistry markers or lung morphology, as measured by histology as described in previous examples herein. However, a dose-related macrophage infiltrate was observed in the lung following aerosol administration. These data demonstrate that antisense oligonucleotides targeted to human IL-4R alpha significantly reduce IL-4R alpha mRNA and protein and IL-4R alpha bio-activity in human pulmonary epithelial cells, and inhalation of an IL-4R alpha antisense oligonucleotide is well-tolerated in mice.

1. A method for prevention, amelioration or treatment of inflammatory respiratory disease comprising (i) selecting a patient diagnosed with inflammatory respiratory disease and (ii) administering to said patient a corticosteroid and an antisense oligonucleotide targeted to IL-4R alpha.

2. A method for prevention, amelioration or treatment of inflammatory respiratory disease in a patient in need of such therapy, comprising (i) selecting a patient being treated with a corticosteroid and (ii) administering to said patient an antisense oligonucleotide targeted to IL-4R alpha.

3. A method for reducing the minimum effective dose of a corticosteroid in a patient diagnosed with inflammatory respiratory disease, comprising (i) selecting a patient being treated with a corticosteroid and (ii) administering to said patient the corticosteroid and an antisense oligonucleotide targeted to IL-4R alpha.

4. A method for improving one or more symptoms associated with inflammatory respiratory disease in a patient, comprising (i) selecting a patient whose disease is not adequately controlled by corticosteroid treatment and (ii) administering to said patient a corticosteroid and an antisense oligonucleotide targeted to IL-4R alpha.

5. A method for improving inflammatory respiratory disease control in a patient, comprising (i) selecting a patient whose disease is not adequately controlled by corticosteroid treatment and (ii) administering to said patient a corticosteroid and an antisense oligonucleotide targeted to IL-4R alpha.

6. The method of claim 1 wherein the inflammatory respiratory disease is asthma, allergic rhinitis, chronic obstructive pulmonary disease or bronchitis.

7. The method of claim 5 wherein the improvement in disease control is measured by a decrease in the number of symptoms, a decrease in the severity of symptoms, a decrease in the duration of symptoms, a decrease in the number of days with symptoms, an inhibition in recurrence of symptoms or a decrease in the dose or frequency of corticosteroid required.

8. The method of claim 4 wherein the symptoms are selected from airway hyperresponsiveness, pulmonary inflammation, mucus accumulation, eosinophil infiltration, increased production of inflammatory cytokines, coughing, sneezing, wheezing, shortness of breath, chest tightness, chest pain, fatigue, runny nose, post-nasal drip, nasal congestion, sore throat, tearing eyes and headache.

9. The method of claim 1 wherein the administering comprises delivery of the corticosteroid and the antisense oligonucleotide in a single formulation.

10. The method of claim 9 wherein delivery of the single formulation is by inhalation.

11. The method of claim 1 wherein the administering comprises delivery of the corticosteroid and the antisense oligonucleotide in separate formulations.

12. The method of claim 11 wherein the separate formulations are delivered simultaneously.

13. The method of claim 11 wherein the separate formulations are delivered at distinct timepoints.

14. The method of claim 11 wherein delivery of one or both formulations is by inhalation.

15. The method of claim 1 wherein the antisense oligonucleotide is 13 to 30 nucleobases in length.

16. The method of claim 15 wherein the antisense oligonucleotide is targeted to at least an 8-nucleobase portion of nucleotides 2056-2087 of human IL-4R alpha (SEQ ID NO: 3).

17. The method of claim 15 wherein the antisense oligonucleotide is targeted to at least an 8-nucleobase portion of nucleotides 2060-2079 of human IL-4R alpha (SEQ ID NO: 3).

18. The method of claim 15 wherein the antisense oligonucleotide comprises SEQ ID NO: 25.

19. The method of claim 15 wherein the nucleotide sequence of the antisense oligonucleotide consists of SEQ ID NO: 25.

20. The method of claim 1 wherein the corticosteroid is budesonide.

21. A pharmaceutical composition comprising a corticosteroid and an antisense oligonucleotide targeted to human IL-4R alpha.

22. The composition of claim 21 wherein the antisense oligonucleotide is 13 to 30 nucleobases in length.

23. The composition of claim 21 wherein the antisense oligonucleotide is targeted to at least an 8-nucleobase portion of nucleotides 2056-2087 of human IL-4R alpha (SEQ ID NO: 3).

24. The composition of claim 21 wherein the antisense oligonucleotide is targeted to at least an 8-nucleobase portion of nucleotides 2060-2079 of human IL-4R alpha (SEQ ID NO: 3).

25. The composition of claim 21 wherein the antisense oligonucleotide comprises SEQ ID NO: 25.

26. The composition of claim 11 wherein the nucleotide sequence of the antisense oligonucleotide consists of SEQ ID NO: 25.

27. The composition of claims 21 wherein said corticosteroid is budesonide.

28. Use of a pharmaceutical composition comprising a corticosteroid and an antisense oligonucleotide targeted to IL-4R alpha for the preparation of a medicament for prevention, amelioration and/or treatment of inflammatory respiratory disease.

29. Use of an antisense oligonucleotide targeted to 1L-4R alpha for the preparation of a medicament for the treatment of inflammatory respiratory disease in a patient being treating with a corticosteroid.

30. Use of an antisense oligonucleotide targeted to IL-4R alpha for the preparation of a medicament for the treatment of inflammatory respiratory disease in a patient whose disease is not adequately controlled by corticosteroid treatment.

31. Use of an antisense oligonucleotide targeted to IL-4R alpha for the preparation of a medicament for reducing the minimum effective dose of a corticosteroid in a patient diagnosed with inflammatory respiratory disease.

32. Use of an antisense oligonucleotide targeted to IL-4R alpha for the preparation of a medicament for reducing the dose of corticosteroid required for prevention, amelioration or treatment of inflammatory respiratory disease.

33. The use of claim 28 wherein the corticosteroid is budesonide.

34. The use of claim 28 wherein the medicament is formulated for delivery by inhalation.

35. The method of claim 7 wherein the symptoms are selected from airway hyperresponsiveness, pulmonary inflammation, mucus accumulation, eosinophil infiltration, increased production of inflammatory cytokines, coughing, sneezing, wheezing, shortness of breath, chest tightness, chest pain, fatigue, runny nose, post-nasal drip, nasal congestion, sore throat, tearing eyes and headache.
